(19) United States
(12) Patent Application Publication
(10) Pub. No.: US 2012/0121508 A1
(43) Pub. Date: May 17, 2012

(54) RADIOLABELED CGRP ANTAGONISTS

(75) Inventors: Ian M. Bell, Harleysville, PA (US); Eric Hostetler, Collegeville, PA (US); Harold G. Selnick, Ambler, PA (US); Craig A. Stump, Pottstown, PA (US)

(73) Assignee: Merck Sharp & Dohme Corp., Rahway, NJ (US)

(21) Appl. No.: 13/386,955

(22) PCT Filed: Jul. 20, 2010
(86) PCT No.: PCT/US10/42509

§ 371 Date: Jan. 25, 2012

Related U.S. Application Data
(60) Provisional application No. 61/228,643, filed on Jul. 27, 2009.

(51) Int. Cl. A61K 51/04; A61P 25/00; C07D 471/10

(52) U.S. Cl. 424/1.81; 544/231; 544/230

(57) ABSTRACT

The present invention is directed to radiolabeled CGRP receptor antagonists which are useful for the quantitative imaging of CGRP receptors in mammals.

### BACKGROUND OF THE INVENTION

Noninvasive, nuclear imaging techniques can be used to obtain basic and diagnostic information about the physiology and biochemistry of a variety of living subjects including experimental animals, normal humans and patients. These techniques rely on the use of sophisticated imaging instrumentation that is capable of detecting radiation emitted from radiotracers administered to such living subjects. The information obtained can be reconstructed to provide planar and tomographic images that reveal distribution of the radiotracer as a function of time. Use of appropriately designed radiotracers can result in images which contain information on the structure, function and most importantly, the physiology and biochemistry of the subject. Much of this information cannot be obtained by other means. The radiotracers used in these studies are designed to have defined behaviors in vivo which permit the determination of specific information concerning the physiology or biochemistry of the subject or the effects that various diseases or drugs have on the physiology or biochemistry of the subject. Currently, radiotracers are available for obtaining useful information concerning such things as cardiac function, myocardial blood flow, lung perfusion, liver function, brain blood flow, regional brain glucose and oxygen metabolism.

Compounds can be labeled with either positron or gamma emitting radionuclides. For imaging, the most commonly used positron emitting (PET) radionuclides are ¹¹C, ¹⁸F, ¹⁵O and ¹³N, all of which are accelerator produced, and have half lifes of 20, 110, 2 and 10 minutes, respectively. Since the half-lives of these radionuclides are so short, it is only feasible to use them at institutions that have an accelerator on site or very close by for their production, thus limiting their use. Several gamma emitting radiotracers are available which can be used by essentially any hospital in the U.S. and in most hospitals worldwide. The most widely used of these are ^{99m}Tc, ²⁰¹Tl and ¹²³I.

In the last two decades, one of the most active areas of nuclear medicine research has been the development of receptor imaging radiotracers. These tracers bind with high affinity and specificity to selective receptors and neuroreceptors. Successful examples include radiotracers for imaging the following receptor systems: estrogen, muscarinic, dopamine D1 and D2, opiate, neuropeptide-Y, cannabinoid-1 and neurokinin-1.

CGRP (Calcitonin Gene-Related Peptide) is a naturally occurring 37-amino acid peptide that is generated by tissue-specific alternate processing of calcitonin messenger RNA and is widely distributed in the central and peripheral nervous system. CGRP is localized predominantly in sensory afferent and central neurons and mediates several biological actions, including vasodilation. CGRP is expressed in alpha- and beta-forms that vary by one and three amino acids in the rat and human, respectively. CGRP-alpha and CGRP-beta display similar biological properties. When released from the cell, CGRP initiates its biological responses by binding to specific cell surface receptors that are predominantly coupled to the activation of adenylyl cyclase. CGRP receptors have been identified and pharmacologically evaluated in several tissues and cells, including those of brain, cardiovascular, endothelial, and smooth muscle origin.

Based on pharmacological properties, these receptors are divided into at least two subtypes, denoted CGRP₁ and CGRP₂. Human α-CGRP-(8-37), a fragment of CGRP that lacks seven N-terminal amino acid residues, is a selective antagonist of CGRP₁, whereas the linear analogue of CGRP, diacetoamido methyl cysteine CGRP ([Cys(ACM)2,7]CGRP), is a selective agonist of CGRP₂. CGRP is a potent neuromodulator that has been implicated in the pathology of cerebrovascular disorders such as migraine and cluster headache. In clinical studies, elevated levels of CGRP in the jugular vein were found to occur during migraine attacks (Goadsby et al., Ann. Neural., 1990, 28, 183-187), salivary levels of CGRP are elevated in migraine subjects between attacks (Bellamy et al., Headache, 2006, 46, 24-33), and CGRP itself has been shown to trigger migrainous headache (Lassen et al., Cephalalgia, 2002, 22, 54-61). In clinical trials, the CGRP antagonist BIBN4096BS has been shown to be effective in treating acute attacks of migraine (Olesen et al., New Engl. J. Med., 2004, 350, 1104-1110) and was able to prevent headache induced by CGRP infusion in a control group (Petersen et al., Clin. Pharmacol. Ther., 2005, 77, 202-213).

CGRP-mediated activation of the trigeminovascular system may play a key role in migraine pathogenesis. Additionally, CGRP activates receptors on the smooth muscle of intracranial vessels, leading to increased vasodilation, which is thought to contribute to headache pain during migraine attacks (Lance, Headache Pathogenesis: Monoamines, Neuropeptides, Purines and Nitric Oxide, Lippincott-Raven Publishers, 1997, 3-9). The middle meningeal artery, the principle artery in the dura mater, is innervated by sensory fibers from the trigeminal ganglion which contain several neuropeptides, including CGRP. Trigeminal ganglion stimulation in the cat resulted in increased levels of CGRP, and in humans, activation of the trigeminal system caused facial flushing and increased levels of CGRP in the external jugular vein (Goadsby et al., Ann. Neurol., 1988, 23, 193-196). Electrical stimulation of the dura mater in rats increased the diameter of the middle meningeal artery, an effect that was blocked by prior administration of CGRP(8-37), a peptide CGRP antagonist (Williamson et al., Cephalalgia, 1997, 17, 525-531). Trigeminal ganglion stimulation increased facial blood flow in the rat, which was inhibited by CGRP(8-37) (Escott et al., Brain Res. 1995, 669, 93-99). Electrical stimulation of the trigeminal ganglion in marmoset produced an increase in facial blood flow that could be blocked by the non-peptide CGRP antagonist BIBN4096BS (Doods et al., Br. J. Pharmacol., 2000, 129, 420-423). Thus the vascular effects of CGRP may be attenuated, prevented or reversed by a CGRP antagonist.

PET (Positron Emission Tomography) radiotracers and imaging technology may provide a powerful method for clinical evaluation and dose selection of CGRP antagonists. The PET tracer of the invention can be used as a research tool to study the interaction of unlabeled CGRP antagonists with CGRP receptors in vivo via competition between the unlabeled drug and the PET tracer for binding to the receptor. These types of studies are useful determining the relationship between CGRP receptor occupancy and dose of unlabelled CGRP antagonist, as well as for studying the duration of blockade of the receptor by various does of unlabeled antagonists, agonists and inverse agonists.

As a clinical tool, the PET tracer of the invention can be used to help define a clinically efficacious does of CGRP receptor antagonists. In animal experiments, the PET tracer can be used to provide information that is useful for choosing between potential drug candidates for selection for clinical development. The PET tracer can also be used to study the regional distribution and concentration of CGRP receptors in living human brain, as well as the brain of living animals and in tissue samples. They can be used to study disease or pharmacologically related changes in CGRP receptor concentrations.

It is, therefore, an object of this invention to develop radiolabeled CGRP receptor antagonists that would be useful not only in traditional exploratory and diagnostic imaging applications, but would also be useful in assays, both in vitro and in vivo, for labeling the CGRP receptor and for competing with unlabeled CGRP receptor antagonists. It is a further object of this invention to develop novel assays which comprise such radiolabeled compounds.

### SUMMARY OF THE INVENTION

The present invention is directed to radiolabeled CGRP receptor antagonists which are useful for the labeling and diagnostic imaging of CGRP receptors in mammals.

### BRIEF DESCRIPTION OF THE DRAWINGS

FIG. 1. FIG. 1A: plasma concentration/receptor occupancy relationship in rhesus monkey brain for the compound of Example 1 using the PET tracer of [¹¹C]-Example 1. A projected plasma level of ˜11 nM of the compound of Example 1 is required to reach 50% CGRP receptor occupancy in rhesus. FIG. 1B: Time-activity curves of the PET of [¹¹C]Example 1 in various regions of rhesus monkey brain before (baseline PET scan: closed symbols) and after (blockade PET scan: open symbols) administration of a CGRP receptor antagonist. Units of tracer uptake are Standardized Uptake Value (SUV) and are normalized for the mass of the subject and injected radioactive dose. The large decrease in SUV of the PET tracer of [¹¹C]Example 1 to homogeneous levels throughout the brain after administration of a CGRP receptor antagonist demonstrates a very high level of CGRP receptor occupancy by the CGRP receptor antagonist.

### DETAILED DESCRIPTION OF THE INVENTION

The invention encompasses a genus of compounds of Formula I

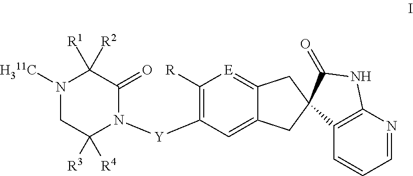

or a pharmaceutically acceptable salt thereof, wherein:

### E is N or CH;

R is H and Y is a linker selected from the group consisting of:

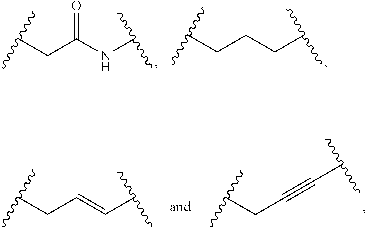

or R and Y together represent

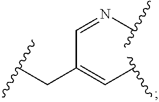

R¹ and R² are independently C₁₋₄alkyl, or R¹ and R² are joined together with the atom to which they are attached to form cycloheptyl, cyclohexyl or cycloheptyl;
and
R³ is hydrogen or methyl and R⁴ is phenyl optionally substituted with 1 to 5 halo groups,
or R³ and R⁴ are joined together with the atom to which they are attached to form cyclopentyl, cyclohexyl or cycloheptyl.

Within the genus, the invention encompasses a subgenus of compounds of Formula Ia

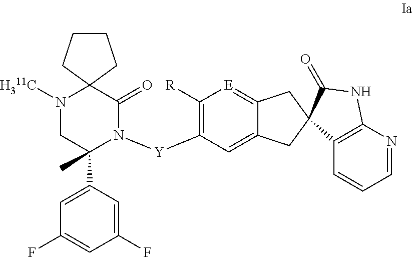

or a pharmaceutically acceptable salt thereof, wherein all other variables are as previously defined.

The invention also encompasses a compound selected from the following table:

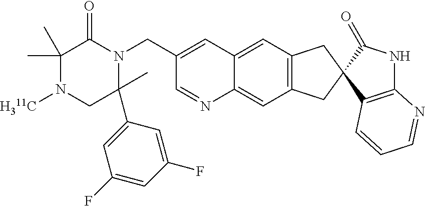

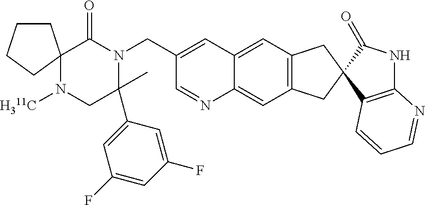

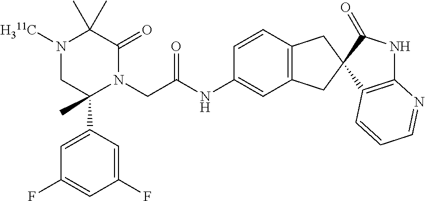

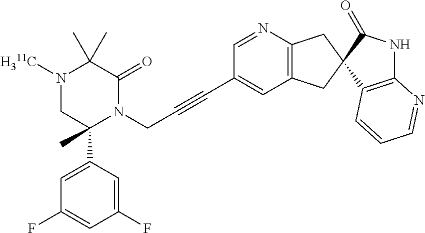

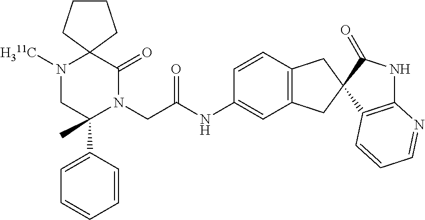

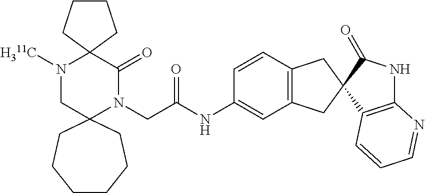

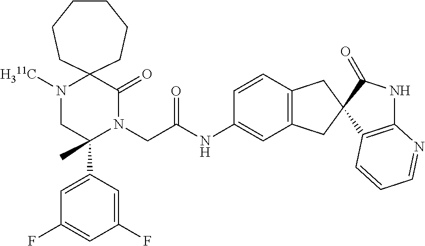

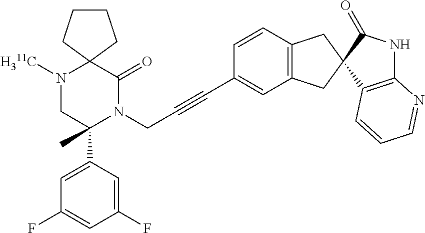

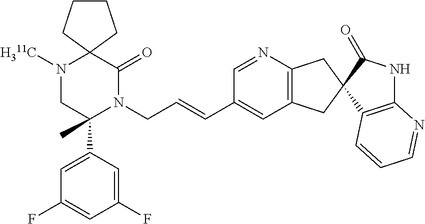

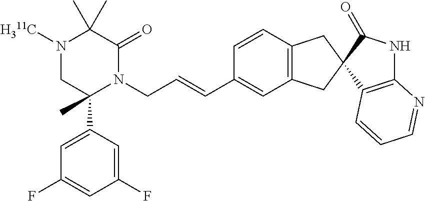

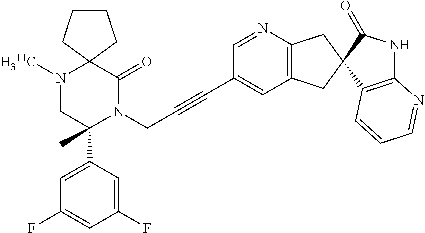

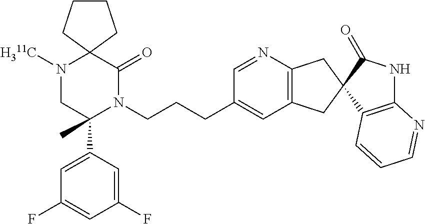

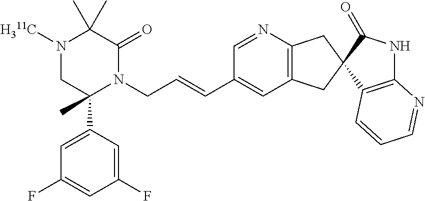

or a pharmaceutically acceptable salt of any of the foregoing.

The invention is also directed to a compound which is

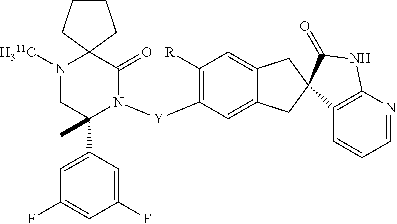

or a pharmaceutically acceptable salt thereof.

The compounds of the invention are a radiolabeled CGRP receptor antagonist which is useful for the quantitative imaging of CGRP receptors in mammals.

An embodiment of the invention encompasses a radiopharmaceutical composition which comprises a compound of Formula I and a pharmaceutically acceptable carrier or excipient.

Another embodiment of the invention encompasses a method for the manufacture of a medicament for the quantitative imaging of CGRP receptors in a mammal which comprises combining a compound of Formula I or a pharmaceutically acceptable salt thereof with a pharmaceutically acceptable carrier or excipient.

Another embodiment of the invention encompasses a method for the quantitative imaging of CGRP receptors in a mammal which comprises administering to the mammal an effective amount of a compound of Formula I, and obtaining an image of CGRP receptors in the mammal using positron emission tomography.

Another embodiment of the invention encompasses a method for the quantitative imaging of the brain in a mammal which comprises administering to the mammal an effective amount of a compound of Formula I, and obtaining an image of the brain in the mammal using positron emission tomography.

Another embodiment of the invention encompasses a method for the quantitative imaging of tissues bearing CGRP receptors in a mammal which comprises administering to the mammal an effective amount of the a compound of Formula I, and obtaining an image of the tissues using positron emission tomography.

Another method for the quantification of CGRP receptors in mammalian tissue which comprises contacting such mammal tissue in which such quantification is desired with an effective amount of a compound of Formula I, and detecting or quantifying the CGRP receptors using positron emission tomography.

The invention also encompasses unlabeled compounds of Formula I or Formula Ia, wherein all variables are defined above. The invention also encompasses the unlabeled compounds described in the following examples or pharmaceutically acceptable salts thereof.

The term “pharmaceutically acceptable salts” refers to salts prepared from pharmaceutically acceptable non-toxic acids including inorganic or organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. It will be understood that, as used herein, references to the compounds of the present invention are meant to also include the pharmaceutically acceptable salts.

In an embodiment of the methods of the present invention, the mammal is a human.

In an embodiment, the compounds of the present invention may be labeled as radiotracers for in vitro imaging. In another embodiment, the compounds of the invention may be prepared as Positron Emission Tomograph (PET) tracers for in vivo imaging and quantification of CGRP receptors.

Radiolabeled CGRP receptor antagonists, when labeled with the appropriate radionuclide, are potentially useful for a variety of in vitro and/or in vivo imaging applications, including diagnostic imaging, basic research, and radiotherapeutic applications. Specific examples of possible diagnostic imaging and radiotherapeutic applications, include determining the location, the relative activity and/or quantifying CGRP receptors, radioimmunoassay of CGRP receptor antagonists, and autoradiography to determine the distribution of CGRP receptors in a mammal or an organ or tissue sample thereof.

In particular, the instant radiolabeled CGRP receptor antagonists are useful for positron emission tomographic (PET) imaging of CGRP receptors in the brain of living humans and experimental animals. These radiolabeled CGRP receptor antagonists may be used as research tools to study the interaction of unlabeled CGRP receptor antagonists with CGRP receptors in vivo via competition between the labeled drug and the radiolabeled compound for binding to the receptor. These types of studies are useful for determining the relationship between CGRP receptor occupancy and dose of unlabeled CGRP receptor antagonist, as well as for studying the duration of blockade of the receptor by various doses of the unlabeled CGRP receptor antagonist, agonists, and inverse agonists. As a clinical tool, the radiolabeled CGRP receptor antagonists may be used to help define a clinically efficacious dose of a CGRP receptor antagonist. In animal experiments, the radiolabeled CGRP receptor antagonists can be used to provide information that is useful for choosing between potential drug candidate for selection for clinical development. The radiolabeled CGRP receptor antagonists may also be used to study the regional distribution and concentration of CGRP receptors in the living human brain, as well as the brain of living experimental animals and in tissue samples. The radiolabeled CGRP receptor antagonists may also be used to study disease or pharmacologically related changes in CGRP receptor concentrations.

For example, positron emission tomography (PET) tracers such as the present radiolabeled CGRP receptor antagonists can be used with currently available PET technology to obtain the following information: relationship between level of receptor occupancy by candidate CGRP receptor antagonists and clinical efficacy in patients; dose selection for clinical trials of CGRP receptor antagonist prior to initiation of long term clinical studies; comparative potencies of structurally novel CGRP receptor antagonists; investigating the influence of CGRP receptor antagonists on in vivo transporter affinity and density during the treatment of clinical targets with CGRP receptor antagonists and other agents; changes in the density and distribution of CGRP receptors during e.g. psychiatric diseases in their active stages, during effective and ineffective treatment and during remission; and changes in CGRP receptor expression and distribution in CNS disorders; imaging neurodegenerative disease where CGRP receptors are involved; and the like.

The radiolabeled CGRP receptor antagonists of the present invention have utility in imaging CGRP receptors or for diagnostic imaging with respect to a variety of disorders associated with CGRP receptors, including one or more of the following conditions or diseases: headache; migraine; cluster headache; chronic tension type headache; pain; chronic pain; neurogenic inflammation and inflammatory pain; neuropathic pain; eye pain; tooth pain; diabetes; non-insulin dependent diabetes mellitus; vascular disorders; inflammation; arthritis; bronchial hyperreactivity, asthma; shock; sepsis; opiate withdrawal syndrome; morphine tolerance; hot flashes in men and women; allergic dermatitis; psoriasis; encephalitis; brain trauma; epilepsy; neurodegenerative diseases; skin diseases; neurogenic cutaneous redness, skin rosaceousness and erythema; inflammatory bowel disease, irritable bowel syndrome, cystitis; and other conditions that may be treated or prevented by antagonism of CGRP receptors. Of particular importance is the acute or prophylactic treatment of headache, including migraine and cluster headache.

For the use of the instant compounds as exploratory or diagnostic imaging agents the radiolabeled compounds may be administered to mammals, preferably humans, in a pharmaceutical composition, either alone or, preferably, in combination with pharmaceutically acceptable carriers or diluents, optionally with known adjuvants, such as alum, in a pharmaceutical composition, according to standard pharmaceutical practice. Such compositions can be administered orally or parenterally, including the intravenous, intramuscular, intraperitoneal, subcutaneous, rectal and topical routes of administration. Preferably, administration is intravenous. Radiotracers labeled with short-lived, positron emitting radionuclides are generally administered via intravenous injection within less than one hour of their synthesis. This is necessary because of the short half-life of the radionuclides involved (20 and 110 minutes for C-11 and F-18 respectively).

The term “composition” as used herein is intended to encompass a product comprising the specified ingredients in the specified amounts, as well as any product which results, directly or indirectly, from combination of the specified ingredients in the specified amounts. Such term in relation to pharmaceutical composition, is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and a pharmaceutically acceptable carrier. By “pharmaceutically acceptable” it is meant the carrier, diluent or excipient must be compatible with the other ingredients of the formulation and not deleterious to the recipient thereof. The terms “administration of” and or “administering a” compound should be understood to mean providing a compound of the invention or a prodrug of a compound of the invention to the patient.

The radiopharmaceutical compositions of this invention may be used in the form of a pharmaceutical preparation, for example, in solid, semisolid or liquid form, which contains one or more of the compound of the present invention, as an active ingredient, in admixture with an organic or inorganic carrier or excipient suitable for external, enteral or parenteral applications. The active ingredient may be compounded, for example, with the usual non-toxic, pharmaceutically acceptable carriers for tablets, pellets, capsules, suppositories, solutions, emulsions, suspensions, and any other form suitable for use. The carriers which can be used are water, glucose, lactose, gum acacia, gelatin, mannitol, starch paste, magnesium trisilicate, talc, corn starch, keratin, colloidal silica, potato starch, urea and other carriers suitable for use in manufacturing preparations, in solid, semisolid, or liquid form, and in addition auxiliary, stabilizing, thickening and coloring agents and perfumes may be used. The active object compound is included in the pharmaceutical composition in an amount sufficient to produce the desired effect upon the process or condition of the disease.

The liquid forms in which the novel compositions of the present invention may be incorporated for administration orally or by injection include aqueous solution, suitably flavoured syrups, aqueous or oil suspensions, and emulsions with acceptable oils such as cottonseed oil, sesame oil, coconut oil or peanut oil, or with a solubilizing or emulsifying agent suitable for intravenous use, as well as elixirs and similar pharmaceutical vehicles. Suitable dispersing or suspending agents for aqueous suspensions include synthetic and natural gums such as tragacanth, acacia, alginate, dextran, sodium carboxymethylcellulose, methylcellulose, polyvinylpyrrolidone or gelatin.

An appropriate dosage level for the unlabeled CGRP receptor antagonist will generally be about 0.01 to 500 mg per kg patient body weight per day which can be administered in single or multiple doses. Preferably, the dosage level will be about 0.1 to about 250 mg/kg per day; more preferably about 0.5 to about 100 mg/kg per day. A suitable dosage level may be about 0.01 to 250 mg/kg per day, about 0.05 to 100 mg/kg per day, or about 0.1 to 50 mg/kg per day. Within this range the dosage may be 0.05 to 0.5, 0.5 to 5 or 5 to 50 mg/kg per day. For oral administration, the compositions are preferably provided in the form of tablets containing 1.0 to 1000 milligrams of the active ingredient, particularly 1.0, 5.0, 10, 15, 20, 25, 50, 75, 100, 150, 200, 250, 300, 400, 500, 600, 750, 800, 900, and 1000 milligrams of the active ingredient for the symptomatic adjustment of the dosage to the patient to be treated. The compounds may be administered on a regimen of 1 to 4 times per day, preferably once or twice per day. This dosage regimen may be adjusted to provide the optimal therapeutic response. It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

When the compounds of the invention are radiolabeled and/or are used as PET tracers, it is preferable that administration be done intravenously. Radiotracers labeled with positron emitting radionuclides are generally administered via intravenous injection within one hour of their synthesis due to the short half-life of the radionuclides involved, which is typically 20 and 110 minutes for C-11 and F-18, respectively. When the radiolabeled CGRP receptor antagonist according to this invention is administered into a human subject, the amount required for imaging will normally be determined by the prescribing physician with the dosage generally varying according to the quantity of emission from the radionuclide. However, in most instances, an effective amount will be the amount of compound sufficient to produce emissions in the range of from about 1-5 mCi.

In one exemplary application, administration occurs in an amount of radiolabeled compound of between about 0.005 μg/kg of body weight to about 50 μg/kg of body weight per day, preferably of between 0.02 μg/kg of body weight to about 3 μg/kg of body weight. The mass associated with a PET tracer is in the form of the natural isotope, for example, ¹²C for an ¹¹C PET tracer and ¹⁹F for an ¹⁸F PET tracer, respectively. A particular analytical dosage that comprises the instant composition includes from about 0.5 μg to about 100 μg of a labeled CGRP receptor antagonist. Preferably, the dosage comprises from about 1 μg to about 50 μg of a radiolabeled CGRP receptor antagonist.

The following illustrative procedure may be utilized when performing PET imaging studies on patients in the clinic. The patient is either unmedicated or premedicated with unlabeled CGRP receptor antagonist or other pharmacological intervention some time prior to the day of the experiment and is fasted for at least 12 hours allowing water intake ad libitum. A 20 G two inch venous catheter is inserted into the contralateral ulnar vein for radiotracer administration. Administration of the PET tracer is often timed to coincide with time of maximum (Tₘₐₓ) or minimum (Tₘₘ) of CGRP receptor antagonist concentration in the blood.

The patient is positioned in the PET camera and a tracer dose of the PET tracer of [¹¹C]Example 1 (<20 mCi) is administered via i.v. catheter. Either arterial or venous blood samples are taken at appropriate time intervals throughout the PET scan in order to analyze and quantitate the fraction of umetabolized PET tracer of [¹¹C]Example 1 in plasma. Images are acquired for up to 120 min. Within ten minutes of the injection of radiotracer and at the end of the imaging session, 1 ml blood samples are obtained for determining the plasma concentration of any unlabeled CGRP receptor antagonist (or other compound of intervention) which may have been administered before the PET tracer.

Tomographic images are obtained through image reconstruction. For determining the distribution of radiotracer, regions of interest (ROIs) are drawn on the reconstructed image including, but not limited to, the striatum, cerebellum and other specific brain regions or areas of the central nervous system. Radiotracer uptakes over time in these regions are used to generate time activity curves (TAC) obtained in the absence of any intervention or in the presence of the unlabeled CGRP receptor antagonist or other compound of intervention at the various dosing paradigms examined. Data are expressed as radioactivity per unit time per unit volume (μCi/cc/mCi injected dose). TAC data are processed with various methods well-known in the field to yield quantitative parameters, such as Binding Potential (BP), that are proportional to the density of unoccupied CGRP receptor. Inhibition of CGRP receptor is then calculated based on the change of BP in the presence of CGRP receptor antagonists at the various dosing paradigms as compared to the BP in the unmedicated state. Inhibition curves are generated by plotting the above data vs the dose (concentration) of CGRP receptor antagonists. The ID₅₀ values are obtained by curve fitting the dose-rate/inhibition curves with equation iii:

B=A₀−A₀*I/(ID₅₀+I)+NS

where B is the %-Dose/g of radiotracer in tissues for each dose of pharmacological intervention, A₀ is the specifically bound radiotracer in a tissue in the absence of a CGRP receptor antagonist, I is the injected dose of antagonist, ID₅₀ is the dose of compound which inhibits 50% of specific radiotracer binding to CGRP receptor, and NS is the amount of non-specifically bond radiotracer.

### Gamma Camera Imaging

Two rats are anesthetized (ketamine/ace-promazine), positioned on the camera head, and their tail veins canulated for ease of injection. One rat is preinjected with an unlabeled CGRP receptor antagonist (10% EtOH/27% PEG/63% H₂O) 30 min, prior to injection of radiotracer to demonstrate non-specific binding. 500 uCi/rat of a PET tracer of [¹¹C]Example 1 is injected via its tail vein, and the catheters flushed with several mis of normal saline. Acquisition of images is started as the radiotracer was injected. Sixty, one minute images are acquired and the rats are subsequently euthanized with sodium pentobarbital. Regions of interest (ROIs) are drawn on the first image which includes the brain, then used to analyze the count rates in subsequent images. ROIs are defined to remain fairly clear during the course of the study, and are assumed to be representative of the entire organ. Count-rates are converted to %-dose/ROI by dividing the count-rate in the ROI by that of the whole rat, which is then multiplied by 100.

### PET Imaging in Dogs

Female beagle dogs weighing 7.7-14.6 kg (11.0±2.3 kg) are premedicated with unlabeled CGRP receptor antagonist (at doses 300, 100, or 30 mg/day) for 2 weeks prior to the day of the experiment and are fasted for at least 12 hours allowing water intake ad libitum. A 20 G two inch venous catheter is placed into the right front leg ulnar vein through which anesthesia is introduced by sodium pentobarbital 25-30 mg/kg in 3-4 ml and maintained with additional pentobarbital at an average dose of 3 mg/kg/hr. Another catheter is inserted into the contralateral ulnar vein for radiotracer administration.

Oxygen saturation of circulating blood is measured with a pulse oximeter (Nellcor Inc., Hayward, Calif.) placed on the tongue of the animal. Circulatory volume is maintained by intravenous infusion of isotonic saline. A 22 G cannula is inserted into the anterior tibial or distal femoral artery for continuous pressure monitoring (Spacelabs™, model 90603A). EKG, heart rate, and core temperature are monitored continuously. In particular, EKG is observed for ST segment changes and arrhythmias.

The animal is positioned in the PET camera and a tracer dose of the PET tracer of [¹¹C]-Example 1 (<20 mCi) is administered via i.v. catheter. Following the acquisition of the total radiotracer image, an infusion is begun of the unlabeled CGRP receptor antagonist at one of three dose rates (0.1, 1 or 10 mpk/day). After infusion for 25 hrs, the PET tracer of [¹¹C]-Example 1 is again injected via the catheter. Images are again acquired for up to 120 min. Within ten minutes of the injection of radiotracer and at the end of the imaging session, 1 ml blood samples are obtained for determining the plasma concentration of test compound. In one imaging session, a dose of 10 mpk another CGRP receptor antagonist is infused over 5 minutes. This dose has been determined to completely block radiotracer binding and thus is used to determine the maximum receptor-specific signal obtained with the PET radiotracer. At the conclusion of the study, animals are recovered and returned to animal housing.

For uninhibited distribution of radiotracer, regions of interest (ROIs) are drawn on the reconstructed image including the brain. These regions are used to generate time activity curves obtained in the absence of test compound or in the presence of test compound at the various infusion doses examined. Data are expressed as radioactivity per unit time per unit volume (μCi/cc/mCi injected dose). Inhibition curves are generated from the data obtained in a region of interest obtained. By this time, clearance of non-specific binding will have reached steady state. The ID₅₀ are obtained by curve fitting the dose-rate/inhibition curves with equation iii, hereinabove.

The activity of the compound in accordance with the present invention as antagonists of CGRP receptor activity may be demonstrated by methodology known in the art. Inhibition of the binding of ¹²⁵I-CGRP to receptors and functional antagonism of CGRP receptors were determined as follows:

NATIVE RECEPTOR BINDING ASSAY: The binding of ¹²⁵I-CGRP to receptors in SK-N-MC cell membranes was carried out essentially as described (Edvinsson et al. (2001) Eur. J. Pharmacol. 415, 39-44). Briefly, membranes (25 μg) were incubated in 1 mL of binding buffer [10 mM HEPES, pH 7.4, 5 mM MgCl₂ and 0.2% bovine serum albumin (BSA)] containing 10 pM ¹²⁵I-CGRP and antagonist. After incubation at room temperature for 3 h, the assay was terminated by filtration through GFB glass fibre filter plates (PerkinElmer) that had been blocked with 0.5% polyethyleneimine for 3 h. The filters were washed three times with ice-cold assay buffer (10 mM HEPES, pH 7.4 and 5 mM MgCl₂), then the plates were air dried. Scintillation fluid (50 μL) was added and the radioactivity was counted on a Topcount (Packard Instrument). Data analysis was carried out by using Prism and the Kᵢ was determined by using the Cheng-Prusoff equation (Cheng & Prusoff (1973) Biochem. Pharmacol. 22, 3099-3108).

RECOMBINANT RECEPTOR: Human CL receptor (Genbank accession number L76380) was subcloned into the expression vector pIREShyg2 (BD Biosciences Clontech) as a 5′NheI and 3′ PmeI fragment. Human RAMP1 (Genbank accession number AJ001014) was subcloned into the expression vector pIRESpuro2 (BD Biosciences Clontech) as a 5′NheI and 3′NotI fragment. HEK 293 cells (human embryonic kidney cells; ATCC #CRL-1573) were cultured in DMEM with 4.5 g/L glucose, 1 mM sodium pyruvate and 2 mM glutamine supplemented with 10% fetal bovine serum (FBS), 100 units/mL penicillin and 100 μg/mL streptomycin, and maintained at 37° C. and 95% humidity. Cells were subcultured by treatment with 0.25% trypsin with 0.1% EDTA in HBSS. Stable cell line generation was accomplished by co-transfecting 10 μg of DNA with 30 μg Lipofectamine 2000 (Invitrogen) in 75 cm² flasks. CL receptor and RAMP1 expression constructs were co-transfected in equal amounts. Twenty-four hours after transfection the cells were diluted and selective medium (growth medium+300 μg/mL hygromycin and 1 μg/mL puromycin) was added the following day. A clonal cell line was generated by single cell deposition utilizing a FACS Vantage SE (Becton Dickinson). Growth medium was adjusted to 150 μg/mL hygromycin and 0.5 μg/mL puromycin for cell propagation.

RECOMBINANT RECEPTOR BINDING ASSAY: Cells expressing recombinant human CL receptor/RAMP1 were washed with PBS and harvested in harvest buffer containing 50 mM HEPES, 1 mM EDTA and Complete protease inhibitors (Roche). The cell suspension was disrupted with a laboratory homogenizer and centrifuged at 48,000 g to isolate membranes. The pellets were resuspended in harvest buffer plus 250 mM sucrose and stored at −70° C. For binding assays, 20 μg of membranes were incubated in 1 ml binding buffer (10 mM HEPES, pH 7.4, 5 mM MgCl₂, and 0.2% BSA) for 3 hours at room temperature containing 10 pM ¹²⁵I-hCGRP (GE Healthcare) and antagonist. The assay was terminated by filtration through 96-well GFB glass fiber filter plates (PerkinElmer) that had been blocked with 0.05% polyethyleneimine. The filters were washed 3 times with ice-cold assay buffer (10 mM HEPES, pH 7.4 and 5 mM MgCl₂). Scintillation fluid was added and the plates were counted on a Topcount (Packard). Non-specific binding was determined and the data analysis was carried out with the apparent dissociation constant (Kᵢ) determined by using a non-linear least squares fitting the bound CPM data to the equation below:

$${Y}_{obsd} = \frac{\begin{matrix}( {Y}_{max} - {Y}_{min} ) ( \% {I}_{max} - \% {I}_{min} / 100 ) + \\ {Y}_{min} + ( {Y}_{max} - {Y}_{min} ) ( 100 - \% {I}_{max} / 100 )\end{matrix}}{1 + ( [ Drug ] / {K}_{i} ( 1 + [ Radiolabel ] / {K}_{d} ) nH}$$

Where Y is observed CPM bound, Yₘₐₓ is total bound counts, Yₘᵢₙ is non specific bound counts, (Yₘₐₓ−Yₘᵢₙ) is specific bound counts, % Iₘₐₓ is the maximum percent inhibition, % 1 min is the minimum percent inhibition, radiolabel is the probe, and the K_{d} is the apparent dissociation constant for the radioligand for the receptor as determined by Hot saturation experiments.

RECOMBINANT RECEPTOR FUNCTIONAL ASSAY: Cells were plated in complete growth medium at 85,000 cells/well in 96-well poly-D-lysine coated plates (Corning) and cultured for ˜19 h before assay. Cells were washed with PBS and then incubated with inhibitor for 30 min at 37° C. and 95% humidity in Cellgro Complete Serum-Free/Low-Protein medium (Mediatech, Inc.) with L-glutamine and 1 g/L BSA. Isobutyl-methylxanthine was added to the cells at a concentration of 300 μM and incubated for 30 min at 37° C. Human α-CGRP was added to the cells at a concentration of 0.3 nM and allowed to incubate at 37° C. for 5 mM. After α-CGRP stimulation the cells were washed with PBS and processed for cAMP determination utilizing the two-stage assay procedure according to the manufacturer's recommended protocol (cAMP SPA direct screening assay system; RPA 559; GE Healthcare). Dose response curves were plotted and IC₅₀ values determined from a 4-parameter logistic fit as defined by the equation y=((a−d)/(1+(x/c)^{b})+d, where y=response, x=dose, a=max response, d=min response, c=inflection point and b=slope.

The present invention is farther directed to a method for the diagnostic imaging of CGRP receptors in a mammal in need thereof which comprises combining a compound of the present invention with a pharmaceutical carrier or excipient.

The synthesis of the compounds of the present invention is illustrated in the following schemes, using the compound of Example 1 as a representative.

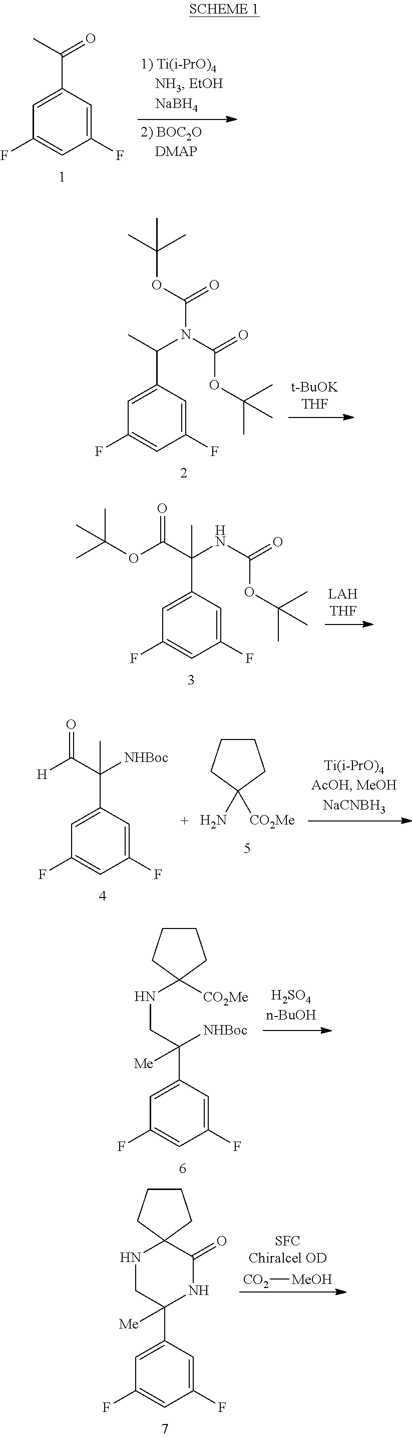

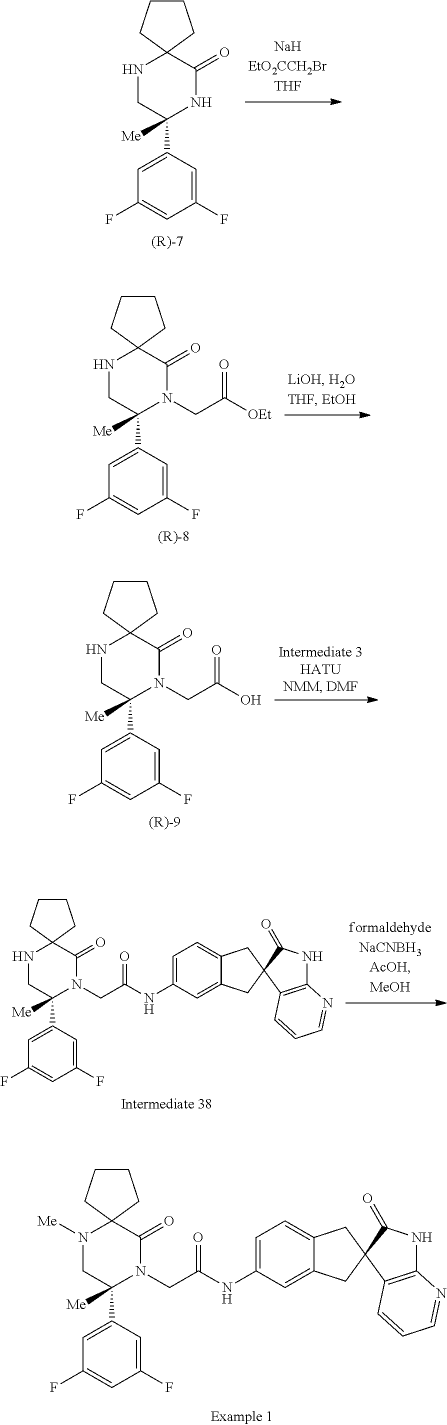

In Scheme 2, the synthesis of the radiotracer compound of [¹¹C]-Example 1 from the precursor Intermediate 38 is shown, as a general illustration of the methodology used herein to prepare such radiolabeled CGRP receptor antagonists.

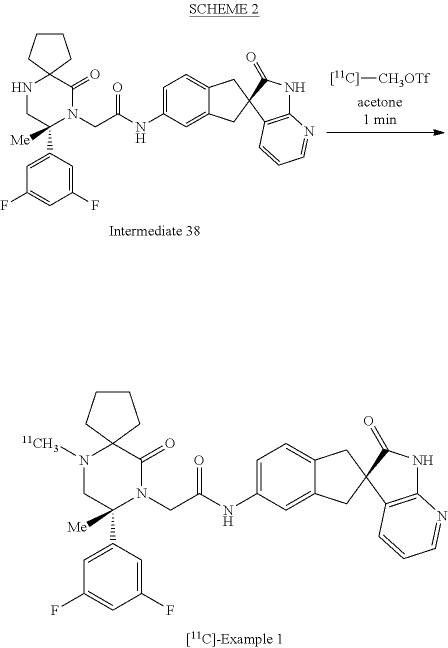

The following examples are provided so that the invention might be more fully understood. These examples are illustrative only and should not be construed as limiting the invention in any way.

### Intermediate 1

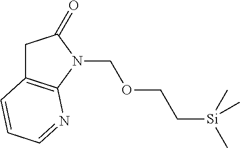

### 1-{[2-(Trimethylsilyl)ethoxy]methyl}-1,3-dihydro-2H-pyrrolo[2,3-b]pyridin-2-one

### Step A. 1-{[2-(Trimethylsilyl)ethoxy]methyl}-1H-pyrrolo[2,3-b]pyridine

Sodium hydride (60% dispersion in mineral oil; 16.2 g, 0.404 mol) was added in portions over 25 min to a solution of 7-azaindole (39.8 g, 0.337 mol) in DMF (200 mL) at 0° C. and the mixture was stirred for 1 h. 2-(Trimethylsilyl)ethoxymethyl chloride (71.8 mL, 0.404 mol) was then added slowly over 15 min, keeping the temperature of the reaction mixture below 10° C. After 1 h, the reaction was quenched with water (500 mL) and the mixture was extracted with CH₂Cl₂ (5×300 mL). The combined organic layers were washed with saturated brine, dried over MgSO₄, filtered, concentrated and dried under high vacuum, to give the title compound. MS: m/z=249 (M+1).

### Step B. 3,3-Dibromo-1-{[2-(trimethylsilyl)ethoxy]methyl}-1,3-dihydro-2H-pyrrolo[2,3-b]pyridin-2-one

A solution of 1-{[2-(trimethylsilyl)ethoxy]methyl}-1H-pyrrolo[2,3-b]pyridine from Step A (43.1 g, 0.1735 mol) in dioxane (300 mL) was added dropwise over 30 min to a suspension of pyridine hydrobromide perbromide (277 g, 0.8677 mol) in dioxane (300 mL). The reaction was stirred at ambient temperature using an overhead mechanical stirrer to produce two layers. After 60 min, the reaction was quenched with water (300 mL) and extracted with EtOAc (500 mL). The aqueous layer was extracted further with EtOAc (2×300 mL) and the combined organic layers were washed with H₂O (4×300 mL; the final wash was pH 5-6), then brine (300 mL), dried over MgSO₄, filtered and concentrated in vacuo. The crude product was immediately dissolved in CH₂Cl₂ and the solution filtered through a plug of silica, eluting with CH₂Cl₂ until the dark red color had completely eluted from the plug. The filtrate was washed with saturated aqueous NaHCO₃ (400 mL), then brine (400 mL), dried over MgSO₄ filtered, and concentrated in vacuo to give the title compound. MS: m/z=423 (M+1).

### Step C. 1-{[2-(Trimethylsilyl)ethoxy]methyl}-1,3-dihydro-2H-pyrrolo[2,3-b]pyridin-2-one

Zinc (100 g, 1.54 mol) was added to a solution of 3,3-dibromo-1-{[2-(trimethylsilyl)ethoxy]methyl}-1,3-dihydro-2H-pyrrolo[2,3-b]pyridin-2-one (65 g, 0.154 mol) in THF (880 mL) and saturated aqueous NH₄Cl (220 mL). After 3 h, the reaction mixture was filtered and concentrated in vacuo. The residue was partitioned between EtOAc and H₂O which resulted in the formation of a white precipitate. Both layers were filtered through a Celite pad and the layers were separated. The aqueous layer was washed with EtOAc (2×500 mL) and the combined organic layers were washed with H₂O, dried over MgSO₄, filtered, and concentrated under reduced pressure. The crude product was purified by silica gel chromatography, eluting with CH₂Cl₂:EtOAc—90:10, to give the title compound. MS: m/z=265 (M+1).

### Intermediate 2

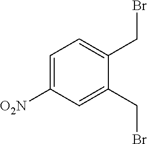

### 1,2-Bis(bromomethyl)-4-nitrobenzene

### Step A. (4-Nitro-1,2-phenylene)dimethanol

A solution of 4-nitrophthalic acid (40 g, 189.5 mmol) in THF (500 mL) was added dropwise over 1.5 h to a solution of borane-THF complex (1 M, 490 mL, 490 mmol), keeping the reaction temperature between 0° C. and 5° C. After the addition, the reaction mixture was allowed to warm slowly to ambient temperature and stirred for 18 h. MeOH (100 mL) was added carefully and the precipitated solid dissolved. The mixture was concentrated in vacuo to about 500 mL, cooled to 0° C., and 10 N NaOH was added to adjust the pH to 10-11. This mixture was extracted with EtOAc (3×600 mL) and the combined organic layers were washed with brine, dried over Na₂SO₄, filtered, and concentrated in vacuo to give the title compound. MS: m/z=207 (M−OH+CH₃CN).

### Step B. 1,2-Bis(bromomethyl)-4-nitrobenzene

Phosphorus tribromide (20.1 mL, 212 mmol) in Et₂O (250 mL) was added dropwise over 1.5 h to a solution of (4-nitro-1,2-phenylene)dimethanol from Step A (35.3 g, 193 mmol) in Et₂O (750 mL). After 18 h, the reaction mixture was cooled to 0° C. and quenched with H₂O (100 mL). The layers were separated and the organic layer was washed with H₂O (2×200 mL), then saturated aqueous NaHCO₃, dried over Na₂SO₄, filtered, and concentrated in vacuo to give the title compound. MS: m/z=309 (M+1).

### Intermediate 3

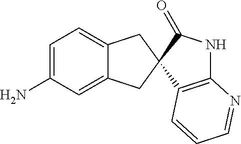

### (R)-5-Amino-1,3-dihydrospiro[indene-2,3′-pyrrolo[2,3-b]pyridin]-2′(1′H)-one

### Step A. (±)-5-Nitro-1′-{[2-(trimethylsilyl)ethoxy]methyl}-1,3-dihydrospiro[indene-2,3′-pyrrolo[2,3-b]pyridin]-2′(1′H)-one

To a solution of 1,2-bis(bromomethyl)-4-nitrobenzene (40.9 g, 132 mmol, described in Intermediate 2) and 1-{[2-(trimethylsilyl)ethoxy]methyl}-1,3-dihydro-2H -pyrrolo[2,3-b]pyridin-2-one (31.5 g, 119 mmol, described in Intermediate 1) in DMF (2 L) was added cesium carbonate (129 g, 397 mmol), portionwise, over 5 min. After 18 h, acetic acid (7.6 mL) was added and the mixture was concentrated to a volume of about 500 mL, then partitioned between EtOAc (1.5 L) and H₂O (1 L). The organic layer was washed with H₂O (1 L), then brine (500 mL), then dried over Na₂SO₄, filtered, and concentrated in vacuo. The crude product was purified by silica gel chromatography, eluting with a gradient of hexane:EtOAc—100:0 to 0:100, to give the title compound. MS: m/z=412 (M+1).

### Step B. (±)-5-Amino-1′-{[2-(trimethylsilyl)ethoxy]methyl}-1,3-dihydrospiro[indene-2,3′-pyrrolo[2,3-b]pyridin]-2′(1′H)-one

A mixture of 10% Pd/C (3 g) and (±)-5-nitro-1′-{[2-(trimethylsilyl)ethoxy]methyl}-1,3-dihydrospiro[indene-2,3′-pyrrolo[2,3-b]pyridin]-2′(1′H)-one from Step A (19.1 g, 46.4 mmol) was stirred vigorously in EtOH (400 mL) under an atmosphere of hydrogen (ca, 1 atm). After 18 h, the mixture was filtered through a pad of Celite, washing extensively with MeOH, and the filtrate was concentrated in vacuo to give the title compound. MS: m/z=382 (M+1).

### Step C. tert-Butyl (R)-(2′-oxo-1′-{[2-(trimethylsilyl)ethoxy]methyl}-1,1′,2′,3-tetrahydrospiro[indene-2,3′-pyrrolo[2,3-b]pyridin]-5-yl)carbamate

A solution of (±)-5-amino-1′-{[2-(trimethylsilyl)ethoxy]methyl}-1,3-dihydrospiro[indene-2,3′-pyrrolo[2,3-b]pyridin]-2′(1′H)-one from Step B (104 g, 273 mmol) and di-tert-butyl dicarbonate (71.5 g, 328 mmol) in CHCl₃ (1 L) was heated to reflux for 17 h. The cooled mixture was concentrated in vacuo and the residue was purified by silica gel chromatography, eluting with hexane:EtOAc—100:0 to 50:50, to give the racemic product. The enantiomers were resolved by HPLC, utilizing a ChiralPak AD column and eluting with EtOH. The first major peak to elute was tert-butyl (S)-(2′-oxo-1′-{[2-(trimethylsilyl)ethoxy]methyl}-1,1′,2′,3-tetrahydrospiro[indene-2,3′-pyrrolo[2,3-b]pyridin]-5-yl)carbamate, and the second major peak to elute was tert-butyl (R)-(2′-oxo-1′-{[2-(trimethylsilyl)ethoxy]methyl}-1,1′,2′,3-tetrahydrospiro[indene-2,3′-pyrrolo[2,3-b]pyridin]-5-yl)carbamate, the title compound. MS: m/z=482 (M+1).

### Step D. (R)-5-Amino-1,3-dihydrospiro[indene-2,3′-pyrrolo[2,3-b]pyridin]-2′(1′H)-one

A solution of tert-butyl (R)-(2′-oxo-1′-{[2-(trimethylsilyl)ethoxy]methyl}-1,1′,2′,3-tetrahydrospiro[indene-2,3′-pyrrolo[2,3-b]pyridin]-5-yl)carbamate from Step C (13.4 g, 27.8 mmol) in MeOH (300 mL) was saturated with HCl (g). The mixture was resaturated with HCl (g) every 30 min until the starting material was consumed, and then concentrated in vacuo. The residue was dissolved in MeOH (150 mL) and treated with ethylenediamine (1.9 mL, 27.8 mmol) and 10 N sodium hydroxide (6 mL, 60 mmol) to adjust the mixture to pH 10. After 30 min, the mixture was diluted with H₂O (400 mL) and extracted with CHCl₃ (1 L). The organic layer was dried over Na₂SO₄, filtered, and concentrated in vacuo. The crude material was triturated with MeOH (35 mL) to give the title compound. MS: m/z=252 (M+1).

### Intermediate 4

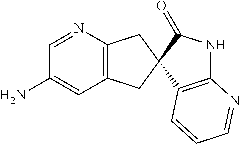

### (6S)-3-Amino-5,7-dihydrospiro[cyclopenta[b]pyridine-6,3′-pyrrolo[2,3-b]pyridin]-2′(1′H)-one

### Step A. (±)-1′-{[2-(Trimethylsilyl)ethoxy]methyl}-3H-spiro[cyclopentane-1,3′-pyrrolo[2,3-b]pyridine]-2′,3(1′H)-dione

To a solution of 1-{[2-(trimethylsilyl)ethoxy]methyl}-1,3-dihydro-2H-pyrrolo[2,3-b]pyridin-2-one (2.50 g, 9.46 mmol, described in Intermediate 1) and cesium carbonate (6.78 g, 20.8 mmol) in DMF (45 mL) was added dropwise a solution of 1,4-dibromobutan-2-one [Meijere et al. (2001) Eur. J. Org. Chem. 20, 3789-3795] (1.59 g, 12.3 mmol) in DMF (45 mL). After 68 h, the mixture was partitioned between Et₂O (200 mL) and H₂O (200 mL). The organic layer was separated and the aqueous layer was further extracted with Et₂O (2×100 mL). The combined organic layers were dried over Na₂SO₄, filtered, and concentrated in vacuo. The crude product was purified by silica gel chromatography, eluting with a gradient of hexane:EtOAc—100:0 to 75:25, to give the title compound. MS: m/z=333 (M+1).

### Step B. (±)-3-Nitro-1′-{[2-(trimethylsilyl)ethoxy]methyl}-5,7-dihydrospiro[cyclopenta[b]pyridine-6,3′-pyrrolo[2,3-b]pyridin]-2′(1′H)-one

A mixture of (±)-1′-{[2-(trimethylsilyl)ethoxy]methyl}-3H-spiro[cyclopentane-1,3′-pyrrolo[2,3-b]pyridine]-2′,3(1′H)-dione from Step A (230 mg, 0.692 mmol) and 1-methyl-3,5-dinitropyridin-2(1H)-one [Tohda et al. (1990) Bull. Chem. Soc. Japan 63, 2820-2827] (173 mg, 0.869 mmol) in 2 M ammonia in MeOH (3.5 mL) was heated to reflux for 18 h. The mixture was concentrated in vacuo and purified by silica gel chromatography, eluting with a gradient of hexane:EtOAc—100:0 to 50:50, to give the title compound. MS: m/z=413 (M+1).

### Step C. (±)-3-Amino-1′-{[2-(trimethylsilyl)ethoxy]methyl}-5,7-dihydrospiro[cyclopenta[b]pyridine-6,3′-pyrrolo[2,3-b]pyridin]-2′(1′H)-one

A mixture of 10% Pd/C (20 mg) and (±)-3-nitro-1′-{[2-(trimethylsilyl)ethoxy]methyl}-5,7-dihydro spiro[cyclopenta[b]pyridine-6,3′-pyrrolo[2,3-b]pyridin]-2′(1′H)-one from Step B (117 mg, 0.284 mmol) was stirred vigorously in MeOH (5 mL) under an atmosphere of hydrogen (ca. 1 atm). After 4.5 h, the mixture was filtered through a pad of Celite, washing extensively with MeOH, and the filtrate was concentrated in vacuo to give the title compound. MS: m/z=383 (M+1).

### Step D. 3-Amino-5,7-dihydrospiro[cyclopenta[b]pyridine-6,3′-pyrrolo[2,3-b]pyridin]-2′(1′H)-one, isomer A

A solution of (±)-3-amino-1′-{[2-(trimethylsilyl)ethoxy]methyl}-5,7-dihydrospiro[cyclopenta[b]pyridine-6,3′-pyrrolo[2,3-b]pyridin]-2′(1′H)-one from Step C (117 mg, 0.306 mmol) in MeOH (5 mL) was saturated with HCl (g). The mixture was stirred for 30 min and then concentrated in vacuo. The residue was dissolved in MeOH (3 mL) and treated with ethylenediamine (0.020 mL, 0.306 mmol) and 10 N sodium hydroxide to adjust the mixture to pH 10. After 1 h, the reaction mixture was purified directly by HPLC using a reversed phase C18 column and eluting with a gradient of H₂O:CH₃CN:CF₃CO₂H—90:10:0.1 to 5:95:0.1. Lyophilization provided the racemic title compound as the TFA salt. The enantiomers were resolved by HPLC, utilizing a ChiralPak AD column and eluting with EtOH. The first major peak to elute was (6S)-3-amino-5,7-dihydrospiro[cyclopenta[b]pyridine-6,3′-pyrrolo[2,3-b]pyridin]-2′(1′H)-one, the title compound, and the second major peak to elute was (6R)-3-amino-5,7-dihydrospiro[cyclopenta[b]pyridine-6,3′-pyrrolo[2,3-b]pyridin]-2′(1′H)-one. MS: m/z=253 (M+1).

### Intermediate 5

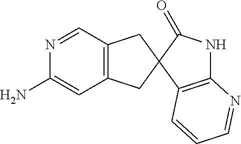

### 3-Amino-5,7-dihydrospiro[cyclopenta[c]pyridine-6,3′-pyrrolo[2,3-b]pyridin]-2′(1′H)-one, isomer A

### Step A. 4,5-Bis(hydroxymethyl)pyridine-2-carbonitrile

To a solution of dimethyl 6-cyanopyridine-3,4-dicarboxylate [Hashimoto et al. (1997) Heterocycles 46, 581] (2.00 g, 9.08 mmol) in EtOH (50 mL) was added lithium borohydride (4.54 mL of a 2 M solution in THF, 9.08 mmol) dropwise. The reaction mixture was stirred at ambient temperature for 3 h, and then cooled to 0° C. Saturated aqueous NaHCO₃ (20 mL) was added slowly and the quenched mixture was extracted with EtOAc (9×100 mL). The combined organic extracts were dried over Na₂SO₄, filtered, and concentrated in vacuo. The crude product was purified by silica gel chromatography, eluting with a gradient of CH₂Cl₂:MeOH—100:0 to 85:15, to give the title compound. MS: m/z=165 (M+1).

### Step B. 4,5-Bis(bromomethyl)pyridine-2-carbonitrile

To a solution of 4,5-bis(hydroxymethyl)pyridine-2-carbonitrile from Step A (750 mg, 4.57 mmol) in THF (15 mL) was added phosphorus tribromide (1.61 g, 5.94 mmol) in THF (5 mL) dropwise. The reaction mixture was stirred at ambient temperature for 2 h, and then cooled to 0° C. Saturated aqueous NaHCO₃ (5 mL) was added slowly and the quenched mixture was extracted with CHCl₃ (2×30 mL). The combined organic extracts were dried over Na₂SO₄, filtered, and concentrated in vacuo. The crude product was purified by silica gel chromatography, eluting with a gradient of hexane:EtOAc—100:0 to 25:75, to give the title compound. MS: m/z=291 (M+1).

### Step C. (±)-2′-Oxo-1′,2′,5,7-tetrahydrospiro[cyclopenta[c]pyridine-6,3′-pyrrolo[2,3-b]pyridine]-3-carbonitrile

To a solution of 4,5-bis(bromomethyl)pyridine-2-carbonitrile from Step B (2.56 g, 8.83 mmol) and 1,3-dihydro-2H-pyrrolo[2,3-b]pyridin-2-one [Marfat & Carta (1987) Tetrahedron Lett. 28, 4027] (1.18 g, 8.83 mmol) in THF (120 mL) and H₂O (60 mL) was added lithium hydroxide monohydrate (1.11 g, 26.5 mmol). After 20 min, the reaction mixture was poured onto water (100 mL) and extracted with EtOAc (3×100 mL). The combined organic extracts were dried over Na₂SO₄, filtered, and concentrated in vacuo. The crude product was purified by silica gel chromatography, eluting with a gradient of CH₂Cl₂:MeOH: NH₄OH—100:0:0 to 95:5:1, to give the title compound. MS: m/z=263 (M+1).

### Step D. (±)-Sodium 2′-oxo-1′,2′,5,7-tetrahydrospiro[cyclopenta[c]pyridine-6,3′-pyrrolo[2,3-b]pyridine]-3-carboxylate

To a solution of (±)-2′-oxo-1′,2′,5,7-tetrahydrospiro[cyclopenta[c]pyridine-6,3′-pyrrolo[2,3-b]pyridine]-3-carbonitrile from Step C (1.53 g, 5.83 mmol) in EtOH (20 mL) was added 5 M aqueous NaOH (3.50 mL). The mixture was heated at reflux for 72 h, with additional 5 M aqueous NaOH (2.00 mL) added at 6 h. The reaction mixture was allowed to cool and was concentrated to dryness in vacuo to afford the title compound in sufficient purity for use in subsequent steps. MS: m/z=282 (M+1).

### Step E. (±)-tert-Butyl (2′-oxo-1′,2′,5,7-tetrahydrospiro[cyclopenta[c]pyridine-6,3′-pyrrolo[2,3-b]pyridin]-3-yl)carbamate

To a suspension of (±)-sodium 2′-oxo-1′,2′,5,7-tetrahydrospiro[cyclopenta[c]pyridine-6,3′-pyrrolo[2,3-b]pyridine]-3-carboxylate from Step D (1.64 g, 5.83 mmol) and triethylamine (1.62 mL, 11.7 mmol) in tert-butanol (50 mL) was added diphenylphosphoryl azide (1.89 mL, 8.75 mmol) and the mixture was heated at reflux for 72 h. Additional diphenylphosphoryl azide (1.89 mL, 8.75 mmol) was added after 24 h and 56 h. The reaction mixture was concentrated in vacuo and then partitioned between CH₂Cl₂ (75 mL) and saturated NaHCO₃ (100 mL). The organic layer was separated and the aqueous layer was further extracted with CH₂Cl₂ (2×50 mL). The combined organic layers were dried over Na₂SO₄, filtered, and concentrated in vacuo. The crude product was purified by silica gel chromatography, eluting with a gradient of CH₂Cl₂:MeOH: NH₄OH—100:0:0 to 95:5:1, to give the title compound. MS: m/z=353 (M+1).

### Step F. 3-Amino-5,7-dihydrospiro[cyclopenta[c]pyridine-6,3′-pyrrolo[2,3-b]pyridin]-2′(1′H)-one, isomer A

A solution of (±)-tert-butyl (2′-oxo-1′,2′,5,7-tetrahydrospiro[cyclopenta[e]pyridine-6,3′-pyrrolo[2,3-b]pyridin]-3-yl)carbamate from Step E (1.39 g, 3.94 mmol) was stirred in CH₂Cl₂ (10 mL) and TFA (3 mL) for 18 h and then concentrated in vacuo to provide the racemic title compound as the TFA salt. The enantiomers were resolved by HPLC, utilizing a ChiralPak AD column and eluting with MeOH. The first major peak to elute was 3-amino-5,7-dihydrospiro[cyclopenta[c]pyridine-6,3′-pyrrolo[2,3-b]pyridin]-2′(1′H)-one, isomer A, the title compound, and the second major peak to elute was 3-amino-5,7-dihydrospiro[cyclopenta[c]pyridine-6,3′-pyrrolo[2,3-b]pyridin]-2′(1′H)-one, isomer B. MS: m/z=253 (M+1).

### Intermediate 6

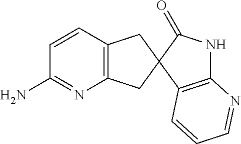

### (±)-2-Amino-5,7-dihydrospiro[cyclopenta[b]pyridine-6,3′-pyrrolo[2,3-b]pyridin]-2′(1′H)-one

### Step A. Dimethyl 6-cyanopyridine-2,3-dicarboxylate

To a solution of dimethylpyridine-2,3-dicarboxylate 1-oxide [Niiyami et al. (2002) Bioorg. Med. Chem. Lett. 12, 3041] (15.3 g, 72.5 mmol) and trimethylsilyl cyanide (15.7 mL, 117 mmol) in DME (161 mL) was added dimethylcarbamoyl chloride (10.5 mL, 114 mmol). The reaction mixture was heated at reflux for 72 h, and then cooled to 0° C. Saturated aqueous NaHCO₃ (800 mL) was added slowly and the quenched mixture was extracted with EtOAc (2×1 L). The combined organic extracts were washed with brine (200 mL), dried over Na₂SO₄, filtered, and concentrated in vacuo. The crude product was purified by silica gel chromatography, eluting with a gradient of hexane:EtOAc—100:0 to 50:50, to give the title compound. MS: m/z=221 (M+1).

### Step B. 5,6-Bis(hydroxymethyl)pyridine-2-carbonitrile

To a solution of dimethyl 6-cyanopyridine-2,3-dicarboxylate from Step A (13.0 g, 59.0 mmol) in EtOH (295 mL) was added lithium borohydride (29.5 mL of a 2 M solution in THF, 59.0 mmol) dropwise. The reaction mixture was stirred at ambient temperature for 4 h, and then cooled to 0° C. Saturated aqueous NaHCO₃ (200 mL) was added slowly and the quenched mixture was extracted with EtOAc (9×100 mL). The combined organic extracts were dried over Na₂SO₄, filtered, and concentrated in vacuo. The crude product was purified by silica gel chromatography, eluting with a gradient of CH₂Cl₂:MeOH—100:0 to 85:15, to give the title compound. MS: m/z=165 (M+1).

### Step C. 5,6-Bis(bromomethyl)pyridine-2-carbonitrile

To a solution of 5,6-bis(hydroxymethyl)pyridine-2-carbonitrile from Step B (2.50 g, 15.2 mmol) in THF (76 mL) was added phosphorus tribromide (5.36 g, 19.8 mmol) in THF (20 mL) dropwise. The reaction mixture was stirred at ambient temperature for 2 h, and then cooled to 0° C. Saturated aqueous NaHCO₃ (20 mL) was added slowly and the quenched mixture was extracted with CH₂Cl₂ (2×200 mL). The combined organic extracts were dried over Na₂SO₄, filtered, and concentrated in vacuo. The crude product was purified by silica gel chromatography, eluting with a gradient of hexane:EtOAc—100:0 to 30:70, to give the title compound. MS: m/z=291 (M+1).

### Step D. (±)-2′-oxo-1′-{[2-(trimethylsilyl)ethoxy]methyl}-1′,2′,5,7-tetrahydrospiro[cyclopenta[b]pyridine-6,3′-pyrrolo[2,3-b]pyridine]-2-carbonitrile

To a solution of 5,6-bis(bromomethyl)pyridine-2-carbonitrile from Step C (1.80 g, 6.21 mmol) and 1-{[2-(trimethylsilyl)ethoxy]methyl}-1,3-dihydro-2H-pyrrolo[2,3-b]pyridin-2-one (1.64 g, 6.21 mmol, described in Intermediate 1) in DMF (207 mL) was added cesium carbonate (6.07 g, 18.6 mmol), portionwise, over 5 min. After 18 h, the mixture was partitioned between CH₂Cl₂ (100 mL), saturated aqueous NaHCO₃ (100 mL) and brine (200 mL). The organic layer was removed and the aqueous layer was extracted further with CH₂Cl₂ (2×100 mL). The combined organic extracts were dried over Na₂SO₄, filtered, and concentrated in vacuo. The crude product was purified by silica gel chromatography, eluting with a gradient of hexane:EtOAc—100:0 to 10:90, to give the title compound, MS: m/z=393 (M+1).

### Step E. (±)-2-Oxo-1′,2′,5,7-tetrahydrospiro[cyclopenta[b]pyridine-6,3′-pyrrolo[2,3-b]pyridine]-2-carboxylic acid

To a solution of (±)-2′-oxo-1′-{[2-(trimethylsilyl)ethoxy]methyl}-1′,2′,5,7-tetrahydrospiro[cyclopenta[b]pyridine-6,3′-pyrrolo[2,3-b]pyridine]-2-carbonitrile from Step D (690 mg, 1.76 mmol) in THF (5 mL) was added 3 N aqueous HCl (36 mL). The mixture was heated at reflux for 18 h, allowed to cool and concentrated to dryness in vacuo. The reaction mixture was dissolved in water (12 mL) and purified directly by HPLC using a reversed phase C18 column and eluting with a gradient of H₂O:CH₃CN:CF₃CO₂H—95:5:0.1 to 5:95:0.1. Lyophilization of the product-containing fractions provided the title compound. MS: m/z=282 (M+1).

### Step F. (±)-tert-Butyl (2′-oxo-1′,2′,5,7-tetrahydrospiro[cyclopenta[b]pyridine-6,3′-pyrrolo[2,3-b]pyridin]-2-yl)carbamate

To a suspension of (±)-2′-oxo-1′,2′,5,7-tetrahydrospiro[cyclopenta[b]pyridine-6,3′-pyrrolo[2,3-b]pyridine]-2-carboxylic acid from Step E (224 mg, 0.796 mmol) and triethylamine (0.333 mL, 2.39 mmol) in tert-butanol (5 mL) was added diphenylphosphoryl azide (0.258 mL, 1.20 mmol) and the mixture was heated at reflux for 1 h. The reaction mixture was concentrated in vacuo and then partitioned between CH₂Cl₂ (20 mL) and saturated NaHCO₃ (20 mL). The organic layer was separated and the aqueous layer was further extracted with CH₂Cl₂ (2×20 mL). The combined organic layers were dried over Na₂SO₄, filtered, and concentrated in vacuo. The crude product was purified by silica gel chromatography, eluting with a gradient of CH₂Cl₂:MeOH: NH₄OH—100:0:0 to 95:5:1, to give the title compound. MS: m/z=353 (M+1).

### Step G. (±)-2-Amino-5,7-dihydrospiro[cyclopenta[b]pyridine-6,3′-pyrrolo[2,3-b]pyridin]-2′(1′H)-one

A solution of (±)-tert-butyl (2′-oxo-1′,2′,5,7-tetrahydrospiro[cyclopenta[b]pyridine-6,3′-pyrrolo[2,3-b]pyridin]-2-yl)carbamate from Step F (147 mg, 0.417 mmol) was stirred in CH₂Cl₂ (6 mL) and TFA (1 mL) for 3 h and then concentrated in vacuo to provide the title compound as the TFA salt. MS: m/z=253 (M+1).

### Intermediate 7

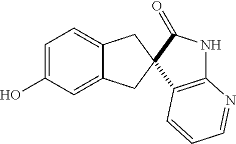

### (R)-5-Hydroxy-1,3-dihydrospiro[indene-2,3′-pyrrolo[2,3-b]pyridin]-2′(1′H)-one

A solution of sodium nitrite (275 mg, 3.98 mmol) in water (1.6 mL) was slowly added to a cooled mixture of (R)-5-amino-1,3-dihydrospiro[indene-2,3′-pyrrolo[2,3-b]pyridin]-2′(1′H)-one (1 g, 3.98 mmol, described in Intermediate 3) in of 10% aqueous H₂SO₄ (8 mL) at 0° C. The ice bath was removed and the reaction allowed to stir at ambient temperature. The reaction mixture was then placed into a 70° C. oil bath and the bath was heated to 100° C. Bubbling was observed and heating was continued until LCMS indicated that the reaction was complete. The reaction was slowly neutralized by addition of 30% NH₄OH (ca. 2 mL) and the precipitate was collected by filtration and washed with water. The solid was then air dried and chromatographed by first mixing with silica and dry loading on a silica gel column. The product was eluted with (10% MeOH/CH₂Cl₂). Concentration of the product containing fractions gave the title compound. MS: m/z=253 (M+1).

### Intermediate 8

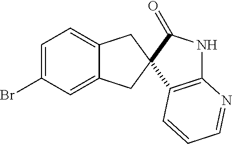

### (R)-5-Bromo-3-dihydrospiro[indene-2,3′-pyrrolo[2,3-b]pyridin]-2′(1′H)-one

To a solution of (R)-5-amino-1,3-dihydrospiro[indene-2,3′-pyrrolo[2,3-b]pyridin]-2′(1′H)-one (500 mg, 1.99 mmol, described in Intermediate 3) in 48% HBr (4 mL) at 0° C. was added slowly over 10 min a solution of sodium nitrite (137 mg, 1.99 mmol) in water (0.8 mL). After 5 minutes CuBr (285 mg, 1.99 mmol) was added and the reaction mixture was placed into a 100° C. oil bath and heated at 100° C. for 20 min. The reaction mixture was then diluted with water followed by 2.5 mL of 30% NH₄OH (2.5 mL) and the resulting solid was collected by filtration and washed with water. The solid was air dried and chromatographed by first mixing with silica and dry loading on a silica gel column. The product was eluted.with (10% MeOH/CH₂Cl₂). Concentration of the product containing fractions gave the title compound. to 740 mg and ˜2 g of silica gel was added. The mixture was dry-loaded on to a silica gel column and the product was eluted with a gradient of EtOAc:hexanes:CH₂Cl₂—10:80:10 to 70:20:10. The product containing fractions were combined and concentrated at reduced pressure to give the title compound. MS: m/z=315 (M+1).

### Intermediate 9

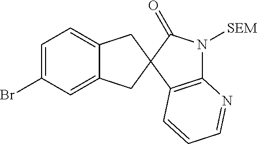

### (±)-5-Bromo-1′-{[2-(trimethylsilyl)ethoxy]methyl}-1,3-dihydrospiro[indene-2,3′-pyrrolo[2,3-b]pyridin]-2′(1′H)-one

To a solution of 1,2-bis(bromomethyl)-4-bromobenzene (40.9 g, 132 mmol) and 1-{[2-(trimethylsilyl)ethoxy]methyl}-1,3-dihydro-2H-pyrrolo[2,3-b]pyridin-2-one (31.5 g, 119 mmol, described in Intermediate 1) in MeOH (2 L) was added cesium carbonate (129 g, 397 mmol), portionwise, over 5 min. After 18 h, the mixture was concentrated to a volume of about 500 mL, then partitioned between EtOAc (1.5 L) and H₂O (1 L). The organic layer was washed with H₂O (1 L), then brine (500 mL), then dried over Na₂SO₄, filtered, and concentrated in vacuo. The crude product was purified by silica gel chromatography, eluting with a gradient of hexane:EtOAc—100:0 to 0:100, to give the title compound. MS: m/z—445 (M+1).

### Intermediate 10

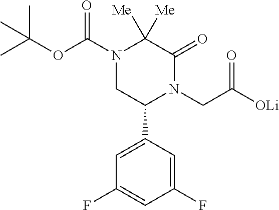

### Lithium [(6R)-4-(tert-butoxycarbonyl)-6-(3,5-difluorophenyl)-3,3-dimethyl-2-oxopiperazin-1-yl]acetate

### Step A. Methyl 2-{[2-(3,5-difluorophenyl)-2-oxoethyl]amino}-2-methylpropanoate

A mixture of methyl α-aminoisobutyrate hydrochloride (10.3 g, 67.0 mmol), 3,5-difluorophenacyl bromide (15.0 g, 63.8 mmol), and K₂CO₃ (17.6 g, 128 mmol) in DMF (100 mL) was stirred at ambient temperature for 3 h. Saturated aqueous NaHCO₃ (400 mL) was added and the mixture was extracted with EtOAc (1 L). The organic layer was washed with brine, dried over Na₂SO₄, filtered, and concentrated in vacuo. The crude product was purified by silica gel chromatography, eluting with a gradient of hexane:EtOAc—100:0 to 0:100, to give the title compound. MS: m/z=272 (M+1).

### Step B. (±)-Ethyl [6-(3,5-difluorophenyl)-3,3-dimethyl-2-oxopiperazin-1-yl]acetate

A mixture of methyl 2-{[2-(3,5-difluorophenyl)-2-oxoethyl]amino}-2-methylpropanoate from Step A (8.60 g, 31.7 mmol), glycine ethyl ester hydrochloride (44.3 g, 317 mmol), and AcOH (5.71 mL, 95 mmol) in MeOH (300 mL) was stirred at ambient temperature for 10 min, NaCNBH₃ (2.39 g, 38.0 mmol) was added and the pH of the mixture was checked and adjusted to pH ˜5 as necessary. The reaction mixture was heated to 50° C. for 18 h. Additional AcOH (4 mL) was added and the reaction mixture was heated to 60° C. for 6 h then allowed to cool and concentrated in vacuo to a volume of ca. 150 mL. The resulting mixture was carefully quenched with saturated aqueous NaHCO₃ (300 mL) and then extracted with CH₂Cl₂ (1 L). The organic extract was washed with brine, dried over Na₂SO₄, filtered, and concentrated in vacuo. The crude product was purified by silica gel chromatography, eluting with hexane:EtOAc—100:0 to 0:100, to give the title compound. MS: m/z=327 (M+1).

### Step C. tert-Butyl (5R)-5-(3,5-difluorophenyl)-4-(2-ethoxy-2-oxoethyl)-2,2-dimethyl-3-oxopiperazine-1-carboxylate

A solution of ethyl [8-(3,5-difluorophenyl)-10-oxo-6,9-diazaspiro[4.5]dec-9-yl]acetate from Step B (2.27 g, 6.96 mmol), N,N-diisopropylethylamine (0.607 mL, 3.48 mmol), and di-tert-butyl dicarbonate (15.2 g, 69.6 mmol) in acetonitrile (30 mL) was stirred at 60° C. for 18 h, then cooled and concentrated under reduced pressure. The crude product was purified by silica gel chromatography, eluting with a gradient of hexane:EtOAc—100:0 to 0:100, to give the racemic product. The enantiomers were separated by SFC, using a Chiralcel OD column and eluting with CO₂:MeOH—80:20. The first major peak to elute was tert-butyl (5S)-5-(3,5-difluorophenyl)-4-(2-ethoxy-2-oxoethyl)-2,2-dimethyl-3-oxopiperazine-1-carboxylate and the second major peak to elute was tert-butyl (5R)-5-(3,5-difluorophenyl)-4-(2-ethoxy-2-oxoethyl)-2,2-dimethyl-3-oxopiperazine-1-carboxylate, the title compound. MS: m/z=371 (M−C₄H₇).

### Step D. Lithium [(6R)-4-(tert-butoxycarbonyl)-6-(3,5-difluorophenyl)-3,3-dimethyl-2-oxopiperazin-1-yl]acetate

To a solution of tert-butyl (5R)-5-(3,5-difluorophenyl)-4-(2-ethoxy-2-oxoethyl)-2,2-dimethyl-3-oxopiperazine-1-carboxylate from Step C (1.18 g, 2.77 mmol) in THF (18 mL) and H₂O (2 mL) was added 1 N aqueous LiOH (3.04 mL, 3.04 mmol) and the resulting mixture was stirred at ambient temperature for 5 h. The mixture was adjusted to pH 6 by addition of 1 N HCl and concentrated to dryness in vacuo to give the title compound. MS: m/z=343 (M−C₄H₇).

### Intermediate 11

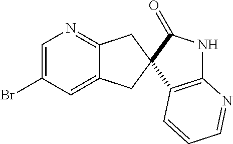

### (6S)-3-Bromo-5,7-dihydrospiro[cyclopenta[b]pyridine-6,3′-pyrrolo[2,3-b]pyridin]-2′(1′H)-one

Essentially following the procedures described for Intermediate 8, but using (6S)-3-amino-5,7-dihydro spiro[cyclopenta[b]pyridine-6,3′-pyrrolo[2,3-b]pyridin]-2′(1′H)-one in place of (R)-5-amino-1,3-dihydrospiro[indene-2,3′-pyrrolo[2,3-b]pyridin]-2′(1′H)-one, the title compound was obtained. MS: m/z=316 (M+1).

### Intermediate 12

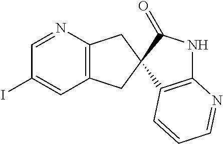

### (6S)-3-Iodo-5,7-dihydrospiro[cyclopenta[b]pyridine-6,3′-pyrrolo[2,3-b]pyridin]-2′(1′H)-one

A solution of sodium nitrite (0.562 g, 8.15 mmol) in water (2 mL) was slowly added to a solution of (6S)-3-amino-5,7-dihydrospiro[cyclopenta[b]pyridine-6,3′-pyrrolo[2,3-b]pyridin]-2′(1′H)-one, bis hydrochloride salt (2.50 g, 7.69 mmol, described in Intermediate 4) in water (12 mL), THF (3 mL) and conc. HCl (1.9 mL) at 0° C. After 30 min, potassium iodide (12.8 g, 77 mmol) was added and the solution stirred an additional 90 min. The reaction mixture was partitioned between CH₂Cl₂:MeOH (100 mL:10 mL) and saturated NaHCO₃ (100 mL). The organic layer was separated and the aqueous layer was further extracted with CH₂Cl₂:MeOH (4×100 mL:10 mL). The combined organic layers were dried over Na₂SO₄, filtered, and concentrated in vacuo directly onto silica gel. The crude product was purified by silica gel chromatography, eluting with a gradient of CH₂Cl₂:MeOH:NH₄OH—100:0:0 to 90:10:1, to give the title compound, MS: m/z=364 (M+1).

### Intermediate 13

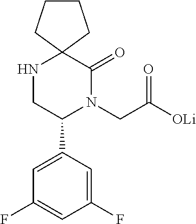

### Lithium [(8R)-8-(3,5-difluorophenyl)-10-oxo-6,9-diazaspiro[4.5]dec-9-yl]acetate

### Step A. Methyl 1-aminocyclopentanecarboxylate hydrochloride

A solution of 1-aminocyclopentanecarboxylic acid (2.00 g, 15.5 mmol) in MeOH (30 mL) was saturated with HCl (g). The resulting mixture was aged at ambient temperature for 2 h and concentrated in vacuo to provide the title compound. MS: m/z=144 (M+1).

### Step B. Methyl 1-{[2-(3,5-difluorophenyl)-2-oxoethyl]amino}cyclopentanecarboxylate

A mixture of methyl 1-aminocyclopentanecarboxylate hydrochloride from Step A (1.50 g, 10.5 mmol), 3,5-difluorophenacyl bromide (3.20 g, 13.6 mmol), and NaHCO₃ (1.32 g, 15.7 mmol) in DMF (30 mL) was stirred at ambient temperature for 6 h. 1 N aqueous HCl (50 mL) was added and the mixture was extracted with EtOAc (75 mL) and this organic extract was discarded. The aqueous layer was adjusted to pH 10 by addition of saturated aqueous Na₂CO₃ (150 mL) and the mixture was extracted with EtOAc (3×75 mL). The combined organic layers were washed with brine, dried over Na₂SO₄, filtered, and concentrated in vacuo. The crude product was purified by HPLC using a reversed phase C18 column and eluting with a gradient of H₂O:CH₃CN:CF₃CO₂H—90:10:0.1 to 5:95:0.1. The product-containing fractions were combined and concentrated to provide the title compound as the TFA salt. MS: m/z=298 (M+1).

### Step C. Ethyl [(8R)-8-(3,5-difluorophenyl)-10-oxo-6,9-diazaspiro[4.5]dec-9-yl]acetate

To a stirred mixture of methyl 1-{[2-(3,5-difluorophenyl)-2-oxoethyl]amino}cyclopentanecarboxylate, TFA salt, from Step B (1.10 g, 2.67 mmol) and glycine ethyl ester hydrochloride (560 mg, 4.01 mmol) in MeOH (7.5 mL) was added N,N-diisopropylethylamine (1.17 mL, 6.69 mmol), followed by AcOH (0.77 mL, 13.4 mmol). The resulting mixture was stirred at ambient temperature for 10 min, then NaCNBH₃ (252 mg, 4.01 mmol) was added. The reaction mixture was heated to 60° C. for 72 h then allowed to cool. The reaction mixture was quenched with saturated aqueous NaHCO₃ and then extracted with EtOAc (3×50 mL). The combined organic extracts were dried over Na₂SO₄, filtered, and concentrated in vacuo. The crude product was purified by HPLC using a reversed phase C18 column and eluting with a gradient of H₂O:CH₃CN:CF₃CO₂H—90:10:0.1 to 5:95:0.1. The product-containing fractions were combined, basified with saturated aqueous NaHCO₃, and extracted with EtOAc. The organic extracts were dried over Na₂SO₄, filtered, and concentrated in vacuo to give the racemic product. The enantiomers were separated by SFC, using a ChiralPak AD column and eluting with CO₂:MeOH—90:10. The first major peak to elute was ethyl [(8S)-8-(3,5-difluorophenyl)-10-oxo-6,9-diazaspiro[4.5]dec-9-yl]acetate, and the second major peak to elute was ethyl [(8R)-8-(3,5-difluorophenyl)-10-oxo-6,9-diazaspiro[4.5]dec-9-yl]acetate, the title compound. MS: m/z=353 (M+1).

### Step D. Lithium [(8R)-8-(3,5-difluorophenyl)-10-oxo-6,9-diazaspiro[4.5]dec-9-yl]acetate

To a solution of ethyl [(8R)-8-(3,5-difluorophenyl)-10-oxo-6,9-diazaspiro[4,5]dec-9-yl]acetate from Step C (90 mg, 0.26 mmol) in THF (3 mL) and H₂O (1 mL) was added 1 N aqueous LiOH (0.31 mL, 0.31 mmol) and the resulting mixture was stirred at ambient temperature for 1 h. The mixture was adjusted to pH 6 by addition of 1 N HCl and concentrated to dryness in vacuo to give the title compound. MS: m/z=325 (M+1).

### Intermediate 14

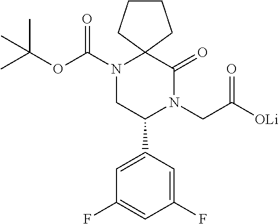

### Lithium [(8R)-6-(tert-butoxycarbonyl)-8-(3,5-difluorophenyl)-10-oxo-6,9-diazaspiro[4.5]dec-9-yl]acetate

### Step A. Methyl 1-{[2-(3,5-difluorophenyl)-2-oxoethyl]amino}cyclopentanecarboxylate

A mixture of methyl 1-aminocyclopentanecarboxylate hydrochloride (10.0 g, 55.7 mmol, described in Intermediate 13), 3,5-difluorophenacyl bromide (14.4 g, 61.2 mmol), and Na₃PO₄ (22.8 g, 139 mmol) in DMF (100 mL) was stirred at ambient temperature for 3.5 h. The reaction mixture was acidified with 1 N aqueous HCl and the mixture was extracted with EtOAc (200 mL) and this organic extract was discarded. The aqueous layer was adjusted to pH 8-9 by addition of saturated aqueous NaHCO₃ and the mixture was extracted with EtOAc (3×250 mL). The combined organic layers were washed with brine, dried over Na₂SO₄, filtered, and concentrated in vacuo. The crude product was purified by silica gel chromatography, eluting with a gradient of hexane:EtOAc—90:10 to 50:50, to give the title compound. MS: m/z=298 (M+1).

### Step B. Ethyl [8-(3,5-difluorophenyl)-10-oxo-6,9-diazaspiro[4.5]dec-9-yl]acetate

A mixture of methyl 1-{[2-(3,5-difluorophenyl)-2-oxoethyl]amino}cyclopentanecarboxylate from Step A (10.0 g, 33.6 mmol), glycine ethyl ester hydrochloride (46.9 g, 336 mmol), and AcOH (5.78 mL, 101 mmol) in MeOH (300 mL) was stirred at ambient temperature for 10 min. NaCNBH₃ (2.54 g, 40.4 mmol) was added and the pH of the mixture was checked and adjusted to pH ˜5 as necessary by addition of AcOH. The reaction mixture was heated to 50° C. for 18 h then allowed to cool. The reaction mixture was carefully quenched with saturated aqueous NaHCO₃ (250 mL) and then extracted with CH₂Cl₂ (3×200 mL). The combined organic extracts were dried over Na₂SO₄, filtered, and concentrated in vacuo. The crude product was purified by silica gel chromatography, eluting with hexane:EtOAc—100:0 to 0:100, to give the title compound. MS: m/z=353 (M+1).

### Step C. tert-Butyl (8R)-8-(3,5-difluorophenyl)-9-(2-ethoxy-2-oxoethyl)-10-oxo-6,9-diazaspiro[4.5]decane-6-carboxylate

A solution of ethyl [8-(3,5-difluorophenyl)-10-oxo-6,9-diazaspiro[4.5]dec-9-yl]acetate from Step B (3.00 g, 8.51 mmol), N,N-diisopropylethylamine (0.743 mL, 4.26 mmol), and di-tert-butyl dicarbonate (9.29 g, 42.6 mmol) in acetonitrile (25 mL) was stirred at 60° C. for 6 h, then cooled and concentrated under reduced pressure. The crude product was purified by silica gel chromatography, eluting with a gradient of hexane:EtOAc—95:5 to 50:50, to give the racemic product. The enantiomers were separated by HPLC, using a Chiralcel OD column and eluting with hexane:i-PrOH:Et₂NH—60:40:0.1. The first major peak to elute was tert-butyl (8S)-8-(3,5-difluorophenyl)-9-(2-ethoxy-2-oxoethyl)-10-oxo-6,9-diazaspiro[4.5]decane-6-carboxylate and the second major peak to elute was tert-butyl (8R)-8-(3,5-difluorophenyl)-9-(2-ethoxy-2-oxoethyl)-10-oxo-6,9-diazaspiro[4.5]decane-6-carboxylate, the title compound. MS: m/z=397 (M−C₄H₇).

### Step D. Lithium [(8R)-6-(tert-butoxycarbonyl)-8-(3,5-difluorophenyl)-10-oxo-6,9-diazaspiro[4.5]dec-9-yl]acetate

To a solution of tert-butyl (8R)-8-(3,5-difluorophenyl)-9-(2-ethoxy-2-oxoethyl)-10-oxo-6,9-diazaspiro[4.5]decane-6-carboxylate from Step C (50 mg, 0.11 mmol) in THF (0.75 mL) and H₂O (0.25 mL) was added 1 N aqueous LiOH (0.12 mL, 0.12 mmol) and the resulting mixture was stirred at ambient temperature for 6 h. The mixture was adjusted to pH 7 by addition of 1 N HCl and concentrated to dryness in vacuo to give the title compound. MS: m/z=369 (M−C₄H₇).

### Intermediate 15

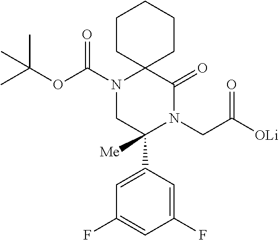

### Lithium [(3R)-1-(tert-butoxycarbonyl)-3-(3,5-difluorophenyl)-3-methyl-5-oxo-1,4-diazaspiro[5.5]undec-4-yl]acetate

### Step A. Di-tert-butyl [1-(3,5-difluorophenyl)ethyl]imidodicarbonate

To a solution of [1-(3,5-difluorophenyl)ethyl]amine (10.0 g, 63.6 mmol) in CH₂Cl₂ (200 mL) at 0° C. was added di-text-butyl dicarbonate (13.9 g, 63.6 mmol) and the resulting mixture was stirred at ambient temperature for 18 h. The solvent was removed under reduced pressure. To the residue was added di-tert-butyl dicarbonate (20.8 g, 95.4 mmol) and DMAP (7.78 g, 63.6 mmol) and the reaction mixture was heated at 80° C. for 2 h. The mixture was allowed to cool and additional di-tert-butyl dicarbonate (69.4 g, 318 mmol) was added. The reaction mixture was heated at 80° C. for 2 h, allowed to cool, and concentrated in vacuo. The crude product was purified by silica gel chromatography, eluting with a gradient of hexane:EtOAc—98:2 to 90:10, to give the title compound. MS: m/z=421 (M+Na+CH₃CN).

### Step B. tert-Bu 12-tert-butoxycarbonyl)amino-2-(3,5-difluorophenyl)propanoate

To a stirred suspension of potassium tert-butoxide in THF (300 mL) at −78° C. was added a solution of di-tert-butyl [1-(3,5-difluorophenyl)ethyl]imidodicarbonate from Step A (22.0 g, 61.6 mmol) in THF (200 mL), dropwise, over 45 min. The reaction mixture was allowed to warm to ambient temperature and stirring was continued for 3 h. The reaction mixture was cooled to −78° C. and quenched with 1 N aqueous HCl (300 mL), warmed to 0° C., and poured into Et₂O (300 mL). The organic layer was extracted and the aqueous layer was extracted further with Et₂O (300 mL). The combined organic extracts were dried over Na₂SO₄, filtered, and concentrated in vacuo. The crude product was purified by silica gel chromatography, eluting with hexane:EtOAc—95:5 to 80:20, to give the title compound. MS: m/z=421 (M+Na+CH₃CN).

### Step C. tert-Butyl [1-(3,5-difluorophenyl)-1-methyl-2-oxoethyl]carbamate

To a stirred solution of tert-butyl 2-[(tert-butoxycarbonyl)amino]-2-(3,5-difluorophenyl)propanoate from Step B (2.00 g, 5.60 mmol) in THF (20 mL) at −78° C. was added LiAlH₄ (5.60 mL of a 1 M solution in THF, 5.60 mmol), dropwise. The reaction mixture was stirred at −78° C. for 6 h, then quenched with EtOAc (5.6 mL), then H₂O (15.6 mL), then 1 N aqueous NaOH (5.6 mL), then EtOAc (17 mL). The reaction mixture was warmed to ambient temperature, stirred for 1 h, filtered, and extracted with EtOAc (2×40 mL). The organic extracts were dried over Na₂SO₄, filtered, and concentrated in vacuo to afford the title compound in sufficient purity for use in the next step. MS: m/z=186 (M−CO₂C₄H₇).

### Step D. Methyl 1-aminocyclohexanecarboxylate hydrochloride

Essentially following the procedures described in Intermediate 13 for methyl 1-aminocyclopentanecarboxylate hydrochloride, but using 1-aminocyclohexanecarboxylic acid in place of 1-aminocyclopentanecarboxylic acid, the title compound was obtained. MS: m/z=158 (M+1).

### Step E. Methyl 1-{[2-[(tert-butoxycarbonyl)amino]-2-(3,5-difluorophenyl)propyl]amino}cyclohexanecarboxylate

A mixture of tert-butyl [1-(3,5-difluorophenyl)-1-methyl-2-oxoethyl]carbamate from Step C (500 mg, 1.75 mmol), methyl 1-aminocyclohexanecarboxylate hydrochloride from Step D (1.38 g, 8.76 mmol), and AcOH (0.301 mL, 5.26 mmol) in MeOH (15 mL) was stirred at ambient temperature for 30 min. NaCNBH₃ (165 mg, 2.63 mmol) was added and the pH of the mixture was checked and adjusted to pH ˜5 as necessary by addition of AcOH. The reaction mixture was stirred at ambient temperature for 1 h, then quenched with saturated aqueous NaHCO₃ (10 mL) and extracted with CH₂Cl₂ (2×50 mL). The combined organic extracts were dried over Na₂SO₄, filtered, and concentrated in vacuo. The crude product was purified by silica gel chromatography, eluting with hexane:EtOAc—100:0 to 80:20, to give the title compound. MS: m/z=427 (M+1).

### Step F. Methyl 1-{[2-amino-2-(3,5-difluorophenyl)propyl]amino}cyclohexanecarboxylate

A solution of methyl 1-{[2-[(tert-butoxycarbonyl)amino]-2-(3,5-difluorophenyl)propyl]amino}cyclohexanecarboxylate from Step E (280 mg, 0.657 mmol) in EtOAc (5 mL) at 0° C. was saturated with HCl (g). The reaction mixture was aged at 0° C. for 30 min, then poured carefully into saturated aqueous NaHCO₃ (10 mL). The resulting mixture was extracted with EtOAc (2×15 mL). The combined organic extracts were dried over Na₂SO₄, filtered, and concentrated in vacuo to give the title compound. MS: m/z=327 (M+1).

### Step G. (3R)-3-(3,5-Difluorophenyl)-3-methyl-1,4-diazaspiro[5,5]undecan-5-one

A solution of methyl 1-{[2-amino-2-(3,5-difluorophenyl)propyl]amino}cyclohexanecarboxylate from Step F (205 mg, 0.628 mmol), and AcOH (0.36 mL, 6.28 mmol) in xylenes (5 mL) was heated at 80° C. for 3 h, allowed to cool, then poured into saturated aqueous NaHCO₃ (5 mL). The resulting mixture was extracted with EtOAc (2×10 mL). The combined organic extracts were dried over Na₂SO₄, filtered, and concentrated under reduced pressure. The crude product was purified by silica gel chromatography, eluting with a gradient of EtOAc:MeOH—100:0 to 92:8, to give the racemic product. The enantiomers were separated by HPLC, using a ChiralPak AD column and eluting with hexane:EtOH:Et₂NH—40:60:0.1. The first major peak to elute was (3R)-3-(3,5-difluorophenyl)-3-methyl-1,4-diazaspiro[5.5]undecan-5-one, the title compound, and the second major peak to elute was (3.3)-3-(3,5-difluorophenyl)-3-methyl-1,4-diazaspiro[5.5]undecan-5-one. MS: m/z=295 (M+1).

### Step H. tert-Butyl (3R)-3-(3,5-difluorophenyl)-3-methyl-5-oxo-1,4-diazaspiro[5.5]undecane-1-carboxylate

A solution of (3R)-3-(3,5-difluorophenyl)-3-methyl-1,4-diazaspiro[5.5]undecan-5-one from Step G (90 mg, 0.306 mmol), N,N-diisopropylethylamine (0.027 mL, 0.153 mmol), and di-tert-butyl dicarbonate (667 mg, 3.06 mmol) in acetonitrile (2 mL) was stirred at 60° C. for 8 h, then cooled and concentrated under reduced pressure. The crude product was purified by silica gel chromatography, eluting with a gradient of hexane:EtOAc—95:5 to 50:50, to give the title compound. MS: m/z=339 (M−C₄H₇).

### Step I. tert-Butyl (3R)-3-(3,5-difluorophenyl)-4-(2-ethoxy-2-oxoethyl)-3-methyl-5-oxo-1,4-diazaspiro[5.5]undecane-1-carboxylate

To a stirred solution of tert-butyl (3R)-3-(3,5-difluorophenyl)-3-methyl-5-oxo-1,4-diazaspiro[5.5]undecane-1-carboxylate from Step H (60 mg, 0.152 mmol) in THF (0.5 mL) at 0° C. was added NaH (12 mg of a 60% dispersion in oil, 030 mmol). After 5 min, ethyl bromoacetate (437 mg, 2.62 mmol) was added and the mixture was allowed to warm to ambient temperature and stirring was continued for 1 h. Saturated aqueous NaHCO₃ (2 mL) was added and the mixture was extracted with EtOAc (2×5 mL). The combined organic layers were dried over Na₂SO₄, filtered, and concentrated in vacuo. The crude product was purified by silica gel chromatography, eluting with a gradient of hexane:EtOAc—95:5 to 60:40, to give the title compound. MS: m/z=425 (M−C₄H₇).

### Step J. Lithium [(3R)-1-(tert-butoxycarbonyl)-3-(3,5-difluorophenyl)-3-methyl-5-oxo-1,4-diazaspiro[5.5]undec-4-yl]acetate

To a solution of tert-butyl (3R)-3-(3,5-difluorophenyl)-4-(2-ethoxy-2-oxoethyl)-3-methyl-5-oxo-1,4-diazaspiro[5.5]undecane-1-carboxylate from Step I (65 mg, 0.135 mmol) in THF (1.5 mL) and H₂O (0.5 mL) was added 1 N aqueous LiOH (0.14 mL, 0.14 mmol) and the resulting mixture was stirred at ambient temperature for 1 h. The mixture was adjusted to pH 7 by addition of 1 N HCl and concentrated to dryness in vacuo to give the title compound. MS: m/z=397 (M−C₄H₇).

### Intermediate 16

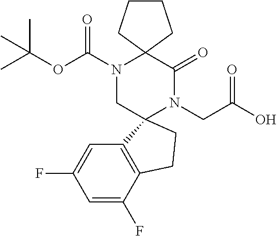

### [(5″R)-1′-(tert-Butoxycarbonyl)-4″,6″-difluoro-3′-oxo-2″,3″-dihydro-4′H-dispiro[cyclopentane-1,2′-piperazine-5′,1″-inden]-4′-yl]acetic acid

### Step A. 4′,6′-Difluoro-2′,3′-dihydro-2H,5H-spiro[imidazolidine-4,1′-indene]-2,5-dione

A mixture of 4,6-difluoroindan-1-one [Musso et al. (2003) J. Med. Chem., 46, 399-408] (14.5 g, 86 mmol), NaCN (12.9 g, 262 mmol), and (NH₄)₂CO₃ (16.8 g, 175 mmol) in H₂O (150 mL) and EtOH (150 mL) was heated at 70° C. for 3 h. Additional (NH₄)₂CO₃ (16.8 g, 175 mmol) was added and heating at 70° C. was continued for 4 h. The mixture was concentrated to dryness under reduced pressure. To the residue was added H₂O (200 mL) and the precipitate was isolated by filtration, washed with H₂O, and dried to give the title compound. MS: m/z=280 (M+1+CH₃CN).

### Step B. 1-Amino-4,6-difluoroindane-1-carboxylic acid hydrochloride

A mixture of 4′,6′-difluoro-2′,3′-dihydro-2H,5H-spiro[imidazolidine-4,1′-indene]-2,5-dione from Step A (16.7 g, 70.1 mmol) and conc. HCl (90 mL) in a high pressure reactor was heated at 180° C. for 5 h. The mixture was cooled to 0° C., vented carefully, and concentrated to dryness in vacuo to afford the title compound. MS: m/z=214 (M+1).

### Step C. Methyl 1-amino-4,6-difluoroindane-1-carboxylate hydrochloride

A solution of 1-amino-4,6-difluoroindane-1-carboxylic acid hydrochloride (2.00 g, 15.5 mmol) in MeOH (100 mL) was saturated with HCl (g). The resulting mixture was heated at reflux for 20 h and concentrated in vacuo to provide the title compound. MS: m/z=228 (M+1).

### Step D. Methyl 1-[(tert-butoxycarbonyl)amino]-4,6-difluoroindane-1-carboxylate

A solution of methyl 1-amino-4,6-difluoroindane-1-carboxylate hydrochloride from Step C (3.82 g, 14.5 mmol), N,N-diisopropylethylamine (5.62 g, 43.5 mmol), and di-tert-butyl dicarbonate (15.8 g, 72.5 mmol) in acetonitrile (40 mL) was stirred at 60° C. for 3 h, then cooled and concentrated under reduced pressure. The crude product was purified by silica gel chromatography, eluting with a gradient of hexane:EtOAc—100:0 to 40:60, to give the title compound. MS: m/z=228 (M−CO₂C₄H₇).

### Step E. tert-Butyl [4,6-difluoro-1-(hydroxymethyl)-2,3-dihydro-1H-inden-1-yl]carbamate

To a stirred solution of methyl 1-[(tert-butoxycarbonyl)amino]-4,6-difluoroindane-1-carboxylate from Step D (2.80 g, 8.55 mmol) in THF (30 mL) at −78° C. was added LiAlH₄ (18.0 mL of a 1 M solution in THF, 18.0 mmol), dropwise, over 30 min. The reaction mixture was stirred at −78° C. for 2 h, then quenched with H₂O (1 mL), then 1 N aqueous NaOH (2 mL), then H₂O (2 mL), then EtOAc (2 mL). The reaction mixture was warmed to ambient temperature, saturated aqueous NaHCO₃ (150 mL) was added, and the mixture was extracted with EtOAc (200 mL). The organic extract was dried over Na₂SO₄, filtered, and concentrated in vacuo. The crude product was purified by silica gel chromatography, eluting with a gradient of hexane:EtOAc—100:0 to 50:50, to give the title compound. MS: m/z 244 (M−C₄H₇).

### Step F. tert-Butyl (4,6-difluoro-1-formyl-2,3-dihydro-1H-inden-1-yl)carbamate

To a stirred solution of oxalyl chloride (0.91 mL, 10.4 mmol) in CH₂Cl₂ (40 mL) at −78° C. was added DMSO (1.48 mL, 20.9 mmol), dropwise, over 5 min. The reaction mixture was stirred for 30 min, during which time it warmed to −60° C., then a solution of tert-butyl [4,6-difluoro-1-(hydroxymethyl)-2,3-dihydro-1H-inden-1-yl]carbamate from Step E (2.08 g, 6.95 mmol) in CH₂Cl₂ (22 mL) was added, dropwise, over 30 min. During the addition, the reaction temperature rose to −45° C. and it was stirred at this temperature for an additional 15 min. To the resulting mixture was added N,N-diisopropylethylamine (7.28 mL, 41.7 mmol), dropwise, over 2 min. The mixture was allowed to warm to 0° C., stirred for 15 min then poured into ice (60 mL) and 1 N aqueous HCl (30 mL). The resulting mixture was extracted with CH₂Cl₂ (2×100 mL). The combined organic extracts were washed with H₂O (30 mL), then brine (50 mL), then dried over Na₂SO₄, filtered, and concentrated in vacuo to give the title compound. MS: m/z=224 (M−OC₄H₉).

### Step G. Methyl 1-[({1-[(tert-butoxycarbonyl)amino]-4,6-difluoro-2,3-dihydro-1H-inden-1-yl}methyl)amino]cyclopentanecarboxylate

A mixture of tert-butyl (4,6-difluoro-1-formyl-2,3-dihydro-1H-inden-1-yl)carbamate from Step F (890 mg, 2.99 mmol), methyl 1-aminocyclopentanecarboxylate (4.25 g, 29.7 mmol, described in Intermediate 25), and AcOH (2.10 mL, 36.7 mmol) in MeOH (32 mL) was stirred at ambient temperature for 20 min. NaCNBH₃ (405 mg, 6.44 mmol) was added and the pH of the mixture was checked and adjusted to pH ˜5 as necessary by addition of AcOH. The reaction mixture was stirred at ambient temperature for 23 h, then quenched with saturated aqueous NaHCO₃ (80 mL) and extracted with EtOAc (200 mL). The organic extract was washed with H₂O (50 mL), dried over Na₂SO₄, filtered, and concentrated in vacuo. The crude product was purified by silica gel chromatography, eluting with hexane:EtOAc—100:0 to 30:70, to give the title compound. MS: m/z=425 (M+1).

### Step H. Methyl 1-{[(1-amino-4,6-difluoro-2,3-dihydro-1H-inden-1-yl)methyl]amino}cyclopentanecarboxylate hydrochloride

A solution of methyl 1-[({1-[(tert-butoxycarbonyl)amino]-4,6-difluoro-2,3-dihydro-1H-inden-1-yl}methyl)amino]cyclopentanecarboxylate from Step G (753 mg, 1.77 mmol) in EtOAc (40 mL) at 0° C. was saturated with HCl (g). The reaction mixture was aged at 0° C. for 45 min then concentrated in vacuo to give the title compound. MS: m/z=325 (M+1),

### Step I. 4″,6″-Difluoro-2″,3″-dihydro-3′H-dispiro[cyclopentane-1,2′-piperazine-5′,1″-inden]-3′-one

A solution of methyl 1-{[(1-amino-4,6-difluoro-2,3-dihydro-1H-inden-1-yl)methyl]amino}cyclopentanecarboxylate hydrochloride from Step H (741 mg, 2.05 mmol), and AcOH (5.0 mL, 6.28 mmol) in xylenes (50 mL) was heated at 150° C. for 24 h, allowed to cool, and concentrated to dryness under reduced pressure. The residue was partitioned between saturated aqueous NaHCO₃ (80 mL) and EtOAc (100 mL). The organic extract was washed with H₂O (60 mL), dried over Na₂SO₄, filtered, and concentrated under reduced pressure to give the title compound. MS: m/z=293 (M+1).

### Step J. tert-Butyl (5″R)-4″,6″-difluoro-3′-oxo-2″,3″-dihydro-1′H-dispiro[cyclopentane-1,2′-piperazine-5′,1″-indene]-1′-carboxylate

A solution of 4″,6″-difluoro-2″,3″-dihydro-3′H-dispiro[cyclopentane-1,2′-piperazine-5′,1″-inden]-3′-one from Step I (453 mg, 1.55 mmol), N,N-diisopropylethylamine (0.135 mL, 0.78 mmol), and di-tert-butyl dicarbonate (3.45 g, 15.8 mmol) in acetonitrile (6 mL) was stirred at 50° C. for 18 h. The reaction mixture was partitioned between saturated aqueous NaHCO₃ (40 mL) and EtOAc (60 mL). The organic extract was dried over Na₂SO₄, filtered, and concentrated under reduced pressure The crude product was purified by silica gel chromatography, eluting with a gradient of hexane:EtOAc—100:0 to 50:50, to give the racemic product. The enantiomers were separated by HPLC, using a ChiralPalc AD column and eluting with hexane:EtOH:Et₂NH—40:60:0.1. The first major peak to elute was tert-butyl (5″R)-4″,6″-difluoro-3′-oxo-2″,3″-dihydro-1′H-dispiro[cyclopentane-1,2′-piperazine-5′,1″-indene]-1′-carboxylate, the title compound, and the second major peak to elute was tert-butyl (5″S)-4″,6″-difluoro-3′-oxo-2″,3″-dihydro-1′H-dispiro[cyclopentane-1,2′-piperazine-5′,1″-indene]-1′-carboxylate. MS: m/z=337 (M−C₄H₇).

### Step K. tert-Butyl (5″R)-4′-(2-ethoxy-2-oxoethyl)-4″,6″-difluoro-3′-oxo-2″,3″-dihydro-1′H-dispiro[cyclopentane-1,2′-piperazine-5′,1″-indene]-1′-carboxylate

To a stirred solution of tert-butyl (5″R)-4″,6″-difluoro-3′-oxo-2″,3″-dihydro-1′H-dispiro[cyclopentane-1,2′-piperazine-5′,1″-indene]-1′-carboxylate from Step J (217 mg, 0.553 mmol) in THF (4 mL) at ambient temperature was added NaH (44 mg of a 60% dispersion in oil, 1.11 mmol). After 15 min, ethyl bromoacetate (185 mg, 1.11 mmol) was added and the mixture was allowed to warm to ambient temperature and stirring was continued for 3 h. Saturated aqueous NaHCO₃ (25 mL) was added and the mixture was extracted with EtOAc (2×30 mL). The combined organic layers were washed with brine, dried over Na₂SO₄, filtered, and concentrated in vacuo. The crude product was purified by silica gel chromatography, eluting with a gradient of hexane:EtOAc—100:0 to 50:50, to give the title compound. MS: m/z=479 (M+1).

### Step L. [(5″R)-1′-(tert-Butoxycarbonyl)-4″,6″-difluoro-3′-oxo-2″,3″-dihydro-4′H-dispiro[cyclopentane-1,2′-piperazine-5′,1″-inden]-4′-yl]acetic acid

To a solution of tert-butyl (5″R)-4′-(2-ethoxy-2-oxoethyl)-4″,6″-difluoro-3′-oxo-2″,3″-dihydro-1′H-dispiro[cyclopentane-1,2′-piperazine-5′,1″-indene]-1′-carboxylate from Step K (258 mg, 0.539 mmol) in THF (3 mL) was added 1 N aqueous LiOH (0.65 mL, 0.65 mmol) and the resulting mixture was stirred at ambient temperature for 20 h. To the reaction mixture was added THF (3 mL), EtOH (0.2 mL), and 1 N aqueous LiOH (0.20 mL, 0.20 mmol) and the resulting mixture was stirred at ambient temperature for 16 h. The mixture was acidified by addition of 1 N aqueous HCl (0.9 mL, 0.9 mmol) and concentrated to dryness in vacuo to give the title compound. MS: m/z=451 (M+1).

### Intermediate 17

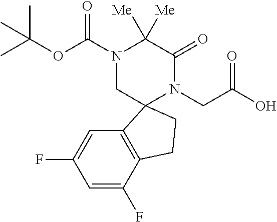

### (±)-[4′-(tert-Butoxycarbonyl)-4,6-difluoro-5′,5′-dimethyl-6′-oxo-2,3-dihydro-1′H-spiro[indene-1,2′-piperazin]-1′-yl]acetic acid

Essentially following the procedures described in Intermediate 16, but using methyl α-aminoisobutyrate in place of methyl 1-aminocyclopentanecarboxylate, the title compound was obtained. MS: m/z=425 (M+1).

### Intermediate 18

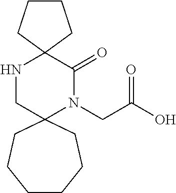

### 16-Oxo-6,15-diazadispiro[4.2.6.2]hexadec-15-yl]acetic acid

Essentially following the procedures described in Intermediate 16, but using methyl 1-aminocycloheptanecarboxylate hydrochloride in place of methyl 1-amino-4,6-difluoroindane-1-carboxylate hydrochloride, the title compound was obtained. MS: m/z=295 (M+1).

### Intermediate 19

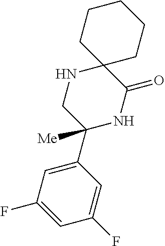

### (3R)-3-(3,5-Difluorophenyl)-3-methyl-1,4-diazaspiro[5.5]undecan-5-one

### Step A. Di-tert-butyl [1-(3,5-difluorophenyl)ethyl]imidodicarbonate

To a solution of [1-(3,5-difluorophenyl)ethyl]amine (10.0 g, 63.6 mmol) in CH₂Cl₂ (200 mL) at 0° C. was added di-tert-butyl dicarbonate (13.9 g, 63.6 mmol) and the resulting mixture was stirred at ambient temperature for 18 h. The solvent was removed under reduced pressure. To the residue was added di-tert-butyl dicarbonate (20.8 g, 95.4 mmol) and DMAP (7.78 g, 63.6 mmol) and the reaction mixture was heated at 80° C. for 2 h. The mixture was allowed to cool and additional di-tert-butyl dicarbonate (69.4 g, 318 mmol) was added. The reaction mixture was heated at 80° C. for 2 h, allowed to cool, and concentrated in mow. The crude product was purified by silica gel chromatography, eluting with a gradient of hexane:EtOAc—98:2 to 90:10, to give the title compound. MS: m/z=421 (M+Na+CH₃CN).

### Step B. tert-Butyl 2-[(tert-butoxycarbonyl)amino]-2-(3,5-difluorophenyl)propanoate

To a stirred suspension of potassium tert-butoxide in THF (300 mL) at −78° C. was added a solution of di-tert-butyl [1-(3,5-difluorophenyl)ethyl]imidodicarbonate from Step A (22.0 g, 61.6 mmol) in THF (200 mL), dropwise, over 45 min. The reaction mixture was allowed to warm to ambient temperature and stirring was continued for 3 h. The reaction mixture was cooled to −78° C. and quenched with 1 N aqueous HCl (300 mL), warmed to 0° C., and poured into Et₂O (300 mL). The organic layer was extracted and the aqueous layer was extracted further with Et₂O (300 mL). The combined organic extracts were dried over Na₂SO₄, filtered, and concentrated in vacuo. The crude product was purified by silica gel chromatography, eluting with hexane:EtOAc—95:5 to 80:20, to give the title compound. MS: m/z=421 (M+Na+CH₃CN).

### Step C. tert-Butyl [1-(3,5-difluorophenyl)-1-methyl-2-oxoethyl]carbamate

To a stirred solution of tert-butyl 2-[(tert-butoxycarbonyl)amino]-2-(3,5-difluorophenyl)propanoate from Step B (2.00 g, 5.60 mmol) in THF (20 mL) at −78° C. was added LiAlH₄ (5.60 mL of a 1 M solution in THF, 5.60 mmol), dropwise. The reaction mixture was stirred at −78° C. for 6 h, then quenched with EtOAc (5.6 mL), then H₂O (15.6 mL), then 1 N aqueous NaOH (5.6 mL), then EtOAc (17 mL). The reaction mixture was warmed to ambient temperature, stirred for 1 h, filtered, and extracted with EtOAc (2×40 mL). The organic extracts were dried over Na₂SO₄, filtered, and concentrated in vacuo to afford the title compound in sufficient purity for use in the next step. MS: m/z=186 (M−CO₂C₄H₇).

### Step D. Methyl 1-aminocyclohexanecarboxylate hydrochloride

Essentially following the procedures described in Intermediate 13 for methyl 1-aminocyclopentanecarboxylate hydrochloride, but using 1-aminocyclohexanecarboxylic acid in place of 1-aminocyclopentanecarboxylic acid, the title compound was obtained. MS: m/z=158 (M+1).

### Step E. Methyl 1-{[2-[(tert-butoxycarbonyl)amino]-2-(3,5-difluorophenyl)propyl]amino}cyclohexanecarboxylate

A mixture of tert-butyl [1-(3,5-difluorophenyl)-1-methyl-2-oxoethyl]carbamate from Step C (500 mg, 135 mmol), methyl 1-aminocyclohexanecarboxylate hydrochloride from Step D (1.38 g, 8.76 mmol), and AcOH (0.301 mL, 5.26 mmol) in MeOH (15 mL) was stirred at ambient temperature for 30 min. NaCNBH₃ (165 mg, 2.63 mmol) was added and the pH of the mixture was checked and adjusted to pH ˜5 as necessary by addition of AcOH. The reaction mixture was stirred at ambient temperature for 1 h, then quenched with saturated aqueous NaHCO₃ (10 mL) and extracted with CH₂Cl₂ (2×50 mL). The combined organic extracts were dried over Na₂SO₄, filtered, and concentrated in vacuo. The crude product was purified by silica gel chromatography, eluting with hexane:EtOAc—100:0 to 80:20, to give the title compound. MS: m/z=427 (M+1).

### Step F. Methyl 1-{[2-amino-2-(3,5-difluorophenyl)propyl]amino}cyclohexanecarboxylate

A solution of methyl 1-{[2-[(tert-butoxycarbonyl)amino]-2-(3,5-difluorophenyl)propyl]amino}cyclohexanecarboxylate from Step E (280 mg, 0.657 mmol) in EtOAc (5 mL) at 0° C. was saturated with HCl (g). The reaction mixture was aged at 0° C. for 30 min, then poured carefully into saturated aqueous NaHCO₃ (10 mL). The resulting mixture was extracted with EtOAc (2×15 mL). The combined organic extracts were dried over Na₂SO₄, filtered, and concentrated in vacuo to give the title compound. MS: m/z=327 (M+1).

### Step G. (3R)-3-(3,5-Difluorophenyl)-3-methyl-1,4-diazaspiro[5.5]undecan-5-one

A solution of methyl 1-{[2-amino-2-(3,5-difluorophenyl)propyl]amino}cyclohexane carboxylate from Step F (205 mg, 0.628 mmol), and AcOH (0.36 mL, 6.28 mmol) in xylenes (5 mL) was heated at 80° C. for 3 h, allowed to cool, then poured into saturated aqueous NaHCO₃ (5 mL). The resulting mixture was extracted with EtOAc (2×10 mL). The combined organic extracts were dried over Na₂SO₄, filtered, and concentrated under reduced pressure. The crude product was purified by silica gel chromatography, eluting with a gradient of EtOAc:MeOH—100:0 to 92:8, to give the racemic product. The enantiomers were separated by HPLC, using a ChiralPak AD column and eluting with hexane:EtOH:Et₂NH—40:60:0.1. The first major peak to elute was (3R)-3-(3,5-difluorophenyl)-3-methyl-1,4-diazaspiro[5.5]undecan-5-one, the title compound, and the second major peak to elute was (3S)-3-(3,5-difluorophenyl)-3-methyl-1,4-diazaspiro[5.5]undecan-5-one. MS: m/z=295 (M+1).

### Intermediate 20

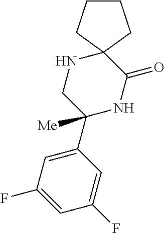

### (8R)-8-(3,5-Difluorophenyl)-8-methyl-6,9-diazaspiro[4.5]decan-10-one

### Step A. [1-(3,5-Difluorophenyl)ethyl]amine

To a stirred mixture of 3′,5′-difluoroacetophenone (86.8 g, 0.556 mol) and 2 M NH₃ in EtOH (1.4 L, 2.8 mol) was added titanium(IV) isopropoxide (326 mL, 1.11 mol) dropwise over 15 min stirring was continued at ambient temperature for 20 h. The mixture was cooled in an ice-water bath and sodium borohydride (31.5 g, 0.834 mol) was added in portions over 60 min. The reaction mixture was stirred for an additional 1 h, and then quenched with aqueous NH₄OH (2 M, 1.3 L) followed by EtOAc (1 L). The resulting mixture was aged for 18 h and filtered through a pad of celite, washing with EtOAc (1 L). To the filtrate was added EtOAc (2 L) and H₂O (1 L) containing NaCl (ca. 100 g). The mixture was shaken and allowed to separate. The organic layer was concentrated in vacuo to a volume of about 500 mL and partitioned between EtOAc (2 L) and saturated aqueous Na₂CO₃ (300 mL). The organic layer was dried (Na₂SO₄), filtered, and concentrated in vacuo to give the title compound. MS: m/z=182 (M+CH₃CN−NH₂).

### Step B. Di-tert-butyl [1-(3,5-difluorophenyl)ethyl]imidodicarbonate

To a solution of [1-(3,5-difluorophenyl)ethyl]amine (76 g, 481 mmol) in CH₂Cl₂ (1 L) at 0° C. was added di-tert-butyl dicarbonate (134 mL, 577 mmol) and the resulting mixture was stirred at ambient temperature for 2 h. The solvent was removed under reduced pressure. To the residue was added di-tert-butyl dicarbonate (336 mL, 1.44 mol) and DMAP (58.8 g, 481 mmol) and the reaction mixture was heated at 60° C. for 18 h. The resulting mixture was heated at 70° C. and additional di-tert-butyl dicarbonate (896 mL, 3.85 mol) was added dropwise, intermittently, over a period of 4 days. The reaction mixture was allowed to cool, and was concentrated in vacuo. The crude product was purified by silica gel chromatography, eluting with a gradient of hexane:EtOAc—100:0 to 85:15, to give the title compound. MS: m/z=421 (M+Na+CH₃CN).

### Step C. tert-Butyl 2-[(tert-butoxycarbonyl)amino]-2-(3,5-difluorophenyl)propanoate

To a stirred suspension of potassium tert-butoxide (64 g, 570 mmol) in THF (800 mL) at −78° C. was added a solution of di-tert-butyl [1-(3,5-difluorophenyl)ethyl]imidodicarbonate (68 g, 190 mmol) in THF (480 mL), dropwise, over 45 min. The reaction mixture was allowed to warm to ambient temperature and stirring was continued for 1 h. The reaction mixture was cooled to −78° C. and quenched with 1 N aqueous HCl (600 mL), warmed to 0° C., and poured into Et₂O (750 mL). The organic layer was extracted and the aqueous layer was extracted further with Et₂O (750 mL). The combined organic extracts were dried over Na₂SO₄, filtered, and concentrated in vacuo. The crude product was purified by silica gel chromatography, eluting with hexane:EtOAc—100:0 to 80:20, to give the title compound. MS: m/z=295 (M+1).

### Step D. tert-Butyl [1-(3,5-difluorophenyl)-1-methyl-2-oxoethyl]carbamate

To a stirred solution of tert-butyl 2-[(tert-butoxycarbonyl)amino]-2-(3,5-difluorophenyl)propanoate (27.0 g, 76 mmol) in THF (350 mL) at −78° C. was added LiAlH₄ (76 mL of a 1 M solution in THF, 76 mmol), dropwise. The reaction mixture was stirred at −78° C. for 3 h, then quenched with EtOAc (76 mL), then H₂O (228 mL), then 1 N aqueous NaOH (76 mL), then EtOAc (228 mL). The reaction mixture was warmed to ambient temperature, stirred for 1 h, filtered, and extracted with EtOAc (2×450 mL). The organic extracts were dried over Na₂SO₄, filtered, and concentrated in vacuo. The crude product was purified by silica gel chromatography, eluting with hexane:EtOAc—100:0 to 70:30, to give the title compound. MS: m/z=186 (M−CO₂C₄H₇).

### Step E. Methyl 1-aminocyclopentanecarboxylate hydrochloride

A solution of 1-aminocyclopentanecarboxylic acid (20.0 g, 155 mmol) in MeOH (300 mL) was saturated with HCl (g), aged for 30 min, and saturated again with HCl (g). The mixture was aged at ambient temperature for 2 h and concentrated to dryness in vacuo. To the white solid was added saturated aqueous NaHCO₃ (350 mL), carefully, with ice cooling, and the resulting mixture was extracted with EtOAc (4×250 mL). The combined organic extracts were dried over Na₂SO₄, filtered, and concentrated in vacuo to give the title compound. MS: m/z=144 (M+1).

### Step F. Methyl 1-{[2-[(tert-butoxycarbonyl)amino]-2-(3,5-difluorophenyl)propyl]amino}cyclopentanecarboxylate

To tert-butyl [1-(3,5-difluorophenyl)-1-methyl-2-oxoethyl]carbamate (28.9 g, 101 mmol) were added methyl 1-aminocyclopentanecarboxylate (43.4 g, 303 mmol) followed by titanium(IV) isopropoxide (44.5 mL, 152 mmol) and the reaction mixture was stirred at ambient temperature for 90 min, diluted with MeOH (130 mL), and cooled in an ice-water bath. To this stirred mixture were added AcOH (29 mL, 507 mmol) followed by NaCNBH₃ (7.64 g, 122 mmol), portionwise, over 5 min. Stirring was continued for 5 min, then the ice-water bath was removed, and stirring was continued for 30 min. The reaction mixture was quenched with saturated aqueous NaHCO₃ (1 L) and extracted with EtOAc (3×1.5 L). The combined organic extracts were washed with brine, dried over Na₂SO₄, filtered, and concentrated in vacuo. The crude product was purified by silica gel chromatography, eluting with hexane:EtOAc—100:0 to 50:50, to give a mixture of the title compound and the corresponding isopropyl ester. MS: m/z 413 (M+1).

### Step G. (8R)-8-(3,5-Difluorophenyl)-8-methyl-6,9-diazaspiro[4.5]decan-10-one

To a solution of methyl 1-{[2-amino-2-(3,5-difluorophenyl)propyl]amino}cyclopentanecarboxylate and the corresponding isopropyl ester (20.1 g, 48.7 mmol) in n-BuOH (1 L) was added c. H₂SO₄ (29 mL, 544 mmol) and the reaction mixture was heated at reflux for 40 h. The cooled mixture was concentrated under reduced pressure to a volume of about 500 mL and then poured into ice-cooled saturated aqueous NaHCO₃ (1 L). The resulting mixture was extracted with EtOAc (2×1 L). The combined organic extracts were dried over Na₂SO₄, filtered, and concentrated under reduced pressure. The crude product was filtered to remove the white precipitate and purified by silica gel chromatography, eluting with a gradient of CHCl₃:MeOH:NH₄OH—100:0:0 to 90:10:0.5, to give some pure fractions of racemic product and some that were contaminated with n-butyl 1-{[2-amino-2-(3,5-difluorophenyl)propyl]amino}cyclopentanecarboxylate. The product from the mixed fractions was recrystallized from EtOAc/Et₂O to give additional racemic product. The enantiomers were separated by SFC, using a Chiralcel OD-H column and eluting with CO₂:MeOH—85:15. The first major peak to elute was (8S)-8-(3,5-difluorophenyl)-8-methyl-6,9-diazaspiro[4.5]decan-10-one and the second major peak to elute was (8R)-8-(3,5-difluorophenyl)-8-methyl-6,9-diazaspiro[4.5]decan-10-one, the title compound. MS: m/z=281 (M+1).

### Intermediate 21

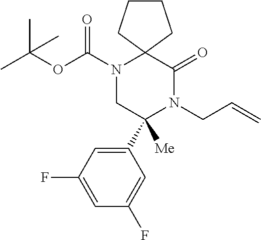

tert-Butyl (8R)-9-allyl-8-(3,5-difluorophenyl)-8-methyl-10-oxo-6,9-diazaspiro[4.5]decane-6-carboxylate

### Step A. tert-Butyl (8R)-8-(3,5-difluorophenyl)-8-methyl-10-oxo-6,9-diazaspiro[4.5]decane-6-carboxylate

A mixture of (8R)-8-(3,5-difluorophenyl)-8-methyl-6,9-diazaspiro[4.5]decan-10-one (5.50 g, 19.6 mmol), N,N-diisopropylethylamine (3.43 mL, 19.6 mmol), and di-tert-butyl dicarbonate (21.4 g, 98 mmol) in acetonitrile (150 mL) was stirred at 60° C. for 18 h, then cooled and concentrated under reduced pressure. The crude product was purified by silica gel chromatography, eluting with a gradient of hexane:EtOAc—95:5 to 50:50, to give the title compound.

### Step B. tert-Butyl (8R)-9-allyl-8-(3,5-difluorophenyl)-8-methyl-10-oxo-6,9-diazaspiro[4.5]decane-6-carboxylate

A solution of tert-butyl (8R)-8-(3,5-difluorophenyl)-8-methyl-10-oxo-6,9-diazaspiro[4.5]decane-6-carboxylate (500 mg, 1.314 mmol) in 2 ml of DMF was added to a suspension of sodium hydride (49.8 mg, 1.971 mmol). When the gas evolution had ceased, allyl bromide (0.171 mL, 1.971 mmol) was added to the ice cooled solution. After 18 hours, the reaction was quenched with brine and extracted with ethyl acetate (3×30 mL). The combined organic layers were washed with brine, dried over Na₂SO₄, filtered and concentrated in vacuo. The oily residue was purified on silica gel, eluting with a gradient of ethyl acetate:hexanes 0:100 to 50:50. The clean fractions were concentrated in vacuo to yield the title compound. MS: m/z=421 (M+1).

### Intermediate 22

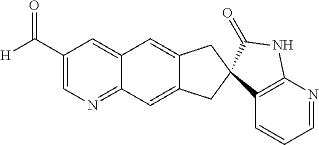

### (7S)-2′-Oxo-1′,2′,6,8-tetrahydrospiro[cyclopenta[g]quinoline-7,3′-pyrrolo[2,3-b]pyridine]-3-carbaldehyde

The title compound was prepared according to known literature methods (International Patent Application Publication No. WO 2007/061677).

The intermediates appearing in the following tables were prepared by analogy to the above intermediates, as described or prepared as a result of similar transformations with modifications known to those skilled in the art. The requisite starting materials were described herein (vide supra), commercially available, known in the literature, or readily synthesized by one skilled in the art. Straightforward protecting group strategies were applied in some routes. In some cases, relevant experimental procedures are indicated in the tables.

TABLE 1

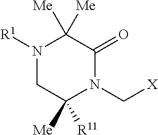

| | | | | | Relevant |
|---|---|---|---|---|---|
| | | | | | experimental |
| Intermediate | R¹ | R¹¹ | X | LCMS (M + 1) | procedures |
| 23 | tert-butoxycarbonyl | 3,5-difluorophenyl | —CO₂H | 435 (M + Na) | Int. 15 |
| 24 | tert-butoxycarbonyl | 3,5-difluorophenyl | —CH═CH₂ | 395 | Int. 21 |
| 25 | H | 3,5-difluorophenyl | —C≡CH | 293 | Int. 21 |

TABLE 2

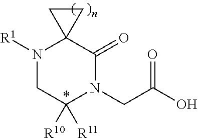

| | | | | | | | Relevant |
|---|---|---|---|---|---|---|---|
| | | | | | | | experimental |
| Intermediate | R¹ | n | R¹⁰ | R¹¹ | * | LCMS (M + 1) | procedures |
| 26 | H | 3 | Me | phenyl | R | 303 | Int. 20 |
| 27 | H | 3 | Me | phenyl | S | 303 | Int. 20 |
| 28 | H | 3 | Me | 3,5-difluorophenyl | R | 339 | Int. 20 |
| 29 | H | 3 | Me | 3,5-difluorophenyl | S | 339 | Int. 20 |
| 30 | tert-butoxycarbonyl | 3 | Me | 3,5-difluorophenyl | R | 383 (M − C₄H₇) | Int. 20 |
| 31 | tert-butoxycarbonyl | 3 | Me | 3,5-difluorophenyl | S | 383 (M − C₄H₇) | Int. 20 |
| 32 | tert-butoxycarbonyl | 5 | Me | 3,5-difluorophenyl | R | 411 (M − C₄H₇) | Int. 15 |
| 33 | tert-butoxycarbonyl | 5 | Me | 3,5-difluorophenyl | S | 411 (M − C₄H₇) | Int. 15 |

### Intermediate 34

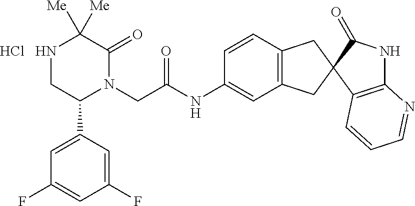

### 2-[(6R)-6-(3,5-Difluorophenyl)-3,3-dimethyl-2-oxopiperazin-1-yl]-N-[(2R)-2′-oxo-1,1′,2′,3-tetrahydrospiro[indene-2,3′-pyrrolo[2,3-b]pyridin]-5-yl]acetamide hydrochloride

To a mixture of lithium [(6R)-4-(tert-butoxycarbonyl)-6-(3,5-difluorophenyl)-3,3-dimethyl-2-oxopiperazin-1-yl]acetate (1.12 g, 277 mmol, described in Intermediate 10), (R)-5-amino-1,3-dihydrospiro[indene-2,3′-pyrrolo[2,3-b]pyridin]-2′(1′H)-one (835 mg, 3.32 mmol, described in Intermediate 3), and HATU (1.26 g, 3.32 mmol) in DMF (12 mL) was added N-methylmorpholine (0.61 mL, 5.54 mmol) and the resulting mixture was stirred at ambient temperature for 90 min. The reaction mixture was diluted with EtOAc (500 mL) and washed successively with 10% citric acid (100 mL), H₂O (100 mL), saturated aqueous NaHCO₃ (100 mL), and brine (100 mL). The organic layer was dried over Na₂SO₄, filtered, and concentrated in vacuo. The residue was purified by silica gel chromatography, eluting with CH₂Cl₂:MeOH—100:0 to 90:10, to give the Boc-protected product. The Boc-protected product was dissolved in EtOAc (75 mL), the solution was cooled to 0° C., and HCl (g) was bubbled in for 2 min. After 15 min, additional HCl (g) was bubbled in for 1 min. The mixture was aged at 0° C. for 30 min and concentrated in vacuo to provide the title compound. MS: m/z 532 (M+1). HRMS: m/z=532.2172; calculated m/z=532.2155 for C₂₉H₂₈F₂N₅O₃.

### Intermediate 35

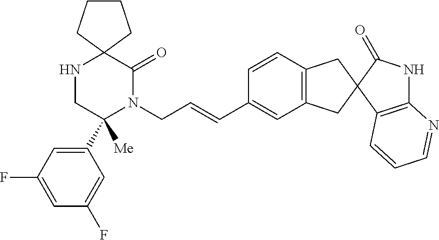

### 5-{(1E)-3-[(8R)-8-(3,5-Difluorophenyl)-8-methyl-10-oxo-6,9-diazaspiro[4.5]dec-9-yl]prop-1-en-1-yl}-1,3-dihydrospiro[indene-2,3′-pyrrolo[2,3-b]pyridin]-2′(1′H)-one, isomer A

### Step A. tert-Butyl (8R)-8-(3,5-difluorophenyl)-8-methyl-10-oxo-9-[(2E)-3-(2′-oxo-1′-{[2-(trimethylsilyl)ethoxy]methyl}-1,1′,2′,3-tetrahydrospiro[indene-2,3′-pyrrolo[2,3-b]pyridin]-5-yl)prop-2-en-1-yl]-6,9-diazaspiro[4.5]decane-6-carboxylate

A suspension of tert-butyl (8R)-9-allyl-8-(3,5-difluorophenyl)-8-methyl-10-oxo-6,9-diazaspiro[4.5]decane-6-carboxylate (0.292 g, 0.694 mmol, described in Intermediate 21) and 5-bromo-1′-{[2-(trimethylsilyl)ethoxy]methyl}-1,3-dihydrospiro[indene-2,3′-pyrrolo[2,3-b]pyridin]-2′(1′H)-one (309 mg, 0.694 mmol, described in Intermediate 9), palladium(II) acetate (46.8 mg, 0.208 mmol), sodium acetate (0.57 mg, 0.694 mmol), and tris-2 methoxy phenyl phosphine (122 mg, 0.347 mmol), in DMF (3 ml) was heated at 130° C. in a microwave reactor for 1 hour. The mixture was filtered through a plug of celite, washing with water and ethyl acetate. The layers were separated, and the aqueous layer was washed with EtOAc (3×30 mL). The combine organic layers were washed with brine, dried over sodium sulfate, filtered and concentrated in vacuo. The residue was purified by silica gel chromatography, eluting with hexanes:EtOAc—100:0 to 50:50, to give the title compound. MS: m/z=785 (M+1).

### Step B. 5-{(1E)-3-[(8R)-8-(3,5-Difluorophenyl)-8-methyl-10-oxo-6,9-diazaspiro[4.5]dec-9-yl]prop-1-en-1-yl}-1,3-dihydrospiro[indene-2,3′-pyrrolo[2,3-b]pyridin]-2′(1′H)-one, isomer A

To a solution of tert-butyl (8R)-8-(3,5-difluorophenyl)-8-methyl-10-oxo-9-[(2E)-3-(2′-oxo-1′-{[2-(trimethylsilyl)ethoxy]methyl}-1,1′,2′,3-tetrahydrospiro[indene-2,3′-pyrrolo[2,3-b]pyridin]-5-yl)prop-2-en-1-yl]-6,9-diazaspiro[4.5]decane-6-carboxylate from Step A (0.2276 g, 0.290 mmol) in CH₂Cl₂ (5 mL) was added TFA (1 mL). The reaction mixture was stirred for 24 hours, then concentrated in vacuo. The residue was dissolved in MeOH (5 mL) and to this solution was added 1 N NaOH (2.90 mL, 2.90 mmol) and ethylenediamine (0.078 mL, 1.160 mmol). The reaction mixture was stirred for 30 min at ambient temperature, then concentrated in vacuo. The residue was partitioned between ethyl acetate and water. The layers were separated and the aqueous layer was washed with ethyl acetate (3×20 mL). The combined organic layers were washed with brine, dried over magnesium sulfate, filtered and concentrated in vacuo to yield the title compound. Diastereomer separation was accomplished by SFC using a Chiralcel OJ column, eluting with CO₂:MeOH—70:30. The second major peak to elute was 5-{(1E)-3-[(8R)-8-(3,5-difluorophenyl)-8-methyl-10-oxo-6,9-diazaspiro[4.5]dec-9-yl]prop-1-en-1-yl}-1,3-dihydrospiro[indene-2,3′-pyrrolo[2,3-b]pyridin]-2′(1′H)-one, isomer B, and the first major peak to elute was 5-{(1E)-3-[(8R)-8-(3,5-difluorophenyl)-8-methyl-10-oxo-6,9-diazaspiro[4.5]dec-9-yl]prop-1-en-1-yl}-1,3-dihydrospiro[indene-2,3′-pyrrolo[2,3-b]pyridin]-2′(1′H)-one, isomer A, the title compound. MS: m/z=555 (M+1). HRMS: m/z=555.2569; calculated m/z=555.2566 for C₃₃H₃₃F₂N₄O₂.

### Intermediate 36

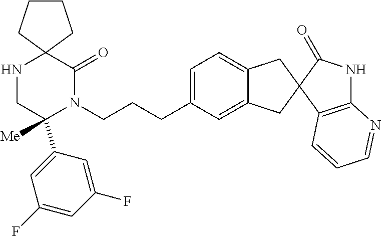

### (2R)-5-{3-[(8R)-8-(3,5-Difluorophenyl)-8-methyl-10-oxo-6,9-diazaspiro[4.5]dec-9-yl]propyl}-1,3-dihydrospiro[indene-2,3′-pyrrolo[2,3-b]pyridin]-2′(1′H)-one, isomer A

5-{(1E)-3 [(8R)-8-(3,5-Difluorophenyl)-8-methyl-10-oxo-6,9-diazaspiro[4.5]dec-9-yl]prop-1-en-1-yl}-1,3-dihydro spiro[indene-2,3′-pyrrolo[2,3-b]pyridin]-2′(1′H)-one, isomer A (32 mg, 0.058 mmol, described in Intermediate 35) was dissolved in MeOH (1 mL) and the solution was passed through 10% Pd/C using an H-Cube™ hydrogenation reactor, eluting with MeOH:AcOH—9:1. The reaction mixture was concentrated in vacuo and partitioned between EtOAc and 10% sodium bicarbonate. The layers were separated and the organic layer was washed with brine, dried over sodium sulfate, filtered and concentrated in vacuo to yield the title compound. MS: m/z=557 (M+1). HRMS: m/z=5572713; calculated m/z=557.2723 for C₃₃H₃₅F₂N₄O₂.

### Intermediate 37

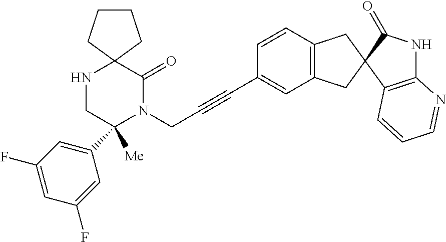

### (2R)-5-{3-[(8R)-8-(3,5-Difluorophenyl)-8-methyl-10-oxo-6,9-diazaspiro[4.5]dec-9-yl]prop-1-yn-1-yl}-1,3-dihydrospiro[indene-2,3′-pyrrolo[2,3-b]pyridin]-2′(1′H)-one

### Step A. (8R)-8-(3,5-Difluorophenyl)-8-methyl-9-prop-2-yn-1-yl-6,9-diazaspiro[4.5]decan-10-one

Sodium hydride (86.0 mg, 2.15 mmol, 60% dispersion in mineral oil) was added to a solution of (8R)-8-(3,5-difluorophenyl)-8-methyl-6,9-diazaspiro[4.5]decan-10-one (502 mg, 1.791 mmol, described in Intermediate 20) in DMF (5 mL). When gas evolution had ceased, propargyl bromide (320 mg, 2.15 mmol, 80 wt % in toluene) was added to the solution at ambient temperature. After 16 hours, the reaction was quenched with water (20 mL) and extracted with CH₂Cl₂ (3×20 mL). The combined organic layers were dried over Na₂SO₄, filtered and concentrated in vacuo. The crude product was purified on silica gel, eluting with a gradient of CH₂Cl₂:EtOAc—98:2 to 50:50, to yield the title compound. MS: m/z=319 (M+1).

### Step B. (2R)-5-{3-[(8R)-8-(3,5-Difluorophenyl)-8-methyl-10-oxo-6,9-diazaspiro[4,5]dec-9-yl]prop-1-yn-1-yl}-1,3-dihydro spire [indene-2,3′-pyrrolo[2,3-b]pyridin]-2′(1′H)-one

A suspension of (8R)-8-(3,5-difluorophenyl)-8-methyl-9-prop-2-yn-1-yl-6,9-diazaspiro[4.5]decan-10-one from Step A (25 mg, 0.079 mmol), (R)-5-bromo-1,3-dihydrospiro[indene-2,3′-pyrrolo[2,3-b]pyridin]-2′(1′H)-one (25 mg, 0.079 mmol described in Intermediate 8), tetrakis(triphenylphosphine)palladium(0) (4.5 mg, 0.0039 mmol), copper(I) iodide (3.0 mg, 0.016 mmol), and triethylamine (0.022 mL, 0.16 mmol) in degassed DMF (0.5 mL) was heated at 80° C. for 16 hours. The mixture was filtered and purified directly by HPLC using a reversed phase C18 column and eluting with a gradient of H₂O:CH₃CN:CF₃CO₂H—90:10:0.1 to 5:95:0.1. The desired fractions were concentrated in vacuo to yield the title compound. MS: m/z=553 (M+1). HRMS: m/z=553.2400; calculated m/z=553.2410 for C₃₃H₃₁F₂N₄O₂.

### Intermediate 38

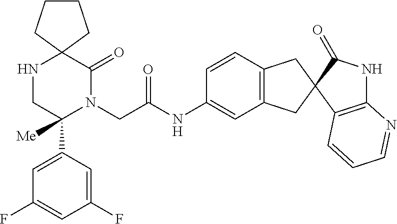

### 2-[(8R)-8-(3,5-Difluorophenyl)-8-methyl-10-oxo-6,9-diazaspiro[4.5]dec-9-yl]-N-[(2R)-2′-oxo-1,1′2′,3-tetrahydrospiro[indene-2,3′-pyrrolo[2,3-b]pyridin]-5-yl]acetamide

To a mixture of [(8R)-8-(3,5-difluorophenyl)-8-methyl-10-oxo-6,9-diazaspiro[4.5]dec-9-yl]acetic acid (3.25 g, 9.61 mmol, described in Intermediate 28), (R)-5-amino-1,3-dihydrospiro[indene-2,3′-pyrrolo[2,3-b]pyridin]-2′(1′H)-one (2.53 g, 10.09 mmol, described in Intermediate 3), and HATU (4.38 g, 11.53 mmol) in DMF (30 mL) was added N-methylmorpholine (2.11 mL, 19.2 mmol) and the resulting mixture was stirred at ambient temperature for 2 h. The reaction mixture was diluted with EtOAc (1 L) and washed successively with saturated aqueous NaHCO₃ (250 mL), and brine (250 mL). The organic layer was dried over Na₂SO₄, filtered, and concentrated in vacuo. The residue was purified by silica gel chromatography, eluting with CH₂Cl₂:MeOH—100:0 to 90:10, to give the title compound. MS: m/z=572 (M+1). HRMS: m/z=572.2447; calculated m/z=572.2468 for C₃₂H₃₂F₂N₅O₃.

### Intermediate 39

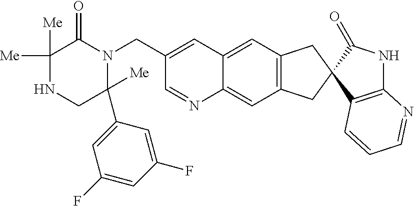

### (7S)-3-{[(2R)-2-(3,5-Difluorophenyl)-2,5,5-trimethyl-6-oxopiperazin-1-yl]methyl}-6,8-dihydrospiro[cyclopenta[g]quinoline-7,3′-pyrrolo[2,3-b]pyridin]-2′(1′H)-one, isomer B

### Step A. (±)-N-[2-[(tert-Butoxycarbonyl)amino]-2-(3,5-difluorophenyl)propyl]-2-methylalanine

To a stirred solution of (±)-tert-butyl [1-(3,5-difluorophenyl)-1-methyl-2-oxoethyl]carbamate (4.00 g, 14.0 mmol, described in U.S. Patent Application Publication No. US 2007/0265225) and 2-methylalanine (4.34 g, 42.1 mmol) in AcOH (25 mL) was added sodium triacetoxyborohydride (3.57 g, 16.8 mmol). The reaction mixture was stirred for 24 h, with additional sodium triacetoxyborohydride (1.00 g) added at 16 and 20 h. The reaction mixture was diluted with water (75 mL) and extracted with CH₂Cl₂ (4×50 mL). The combined organic extracts were dried over Na₂SO₄, filtered, and concentrated in vacuo. The crude product was purified by silica gel chromatography, eluting with a gradient of CH₂Cl₂:MeOH:NH₄OH—97:3:1 to 85:15:1, to give the title compound. MS: m/z=373 (M+1).

### Step B. (±)-N-[2-Amino-2-(3,5-difluorophenyl)propyl]-2-methylalanine

A solution of the (±)-N-[2-[(tert-butoxycarbonyl)amino]-2-(3,5-difluorophenyl)propyl]-2-methylalanine from Step A (878 mg, 2.36 mmol) in CH₂Cl₂ (9 mL) and CF₃CO₂H (3 mL) was aged at ambient temperature for 3 h. The reaction mixture was concentrated in vacuo to give the title compound as the trifluoroacetate salt. MS: m/z=273 (M+1).

### Step C. N-[2-(3,5-Difluorophenyl)-2-({[(7S)-2′-oxo-1′,2′,6,8-tetrahydrospiro[cyclo-penta[g]quinoline-7,3′-pyrrolo[2,3-b]pyridin]-3-yl]methyl}amino)propyl]-2-methylalanine

To a stirred solution of (7S)-2′-oxo-1,2′,6,8-tetrahydrospiro[cyclopenta[g]quinoline-7,3′-pyrrolo[2,3-b]pyridine]-3-carbaldehyde (150 mg, 0.476 mmol, described in Intermediate 1), (±)-N-[2-amino-2-(3,5-difluorophenyl)propyl]-2-methylalanine trifluoroacetate from Step B (238 mg, 0.476 mmol), and AcOH (0.136 mL, 2.38 mmol) in DCE (3 mL) was added sodium triacetoxyborohydride (121 mg, 0.571 mmol). The reaction mixture was stirred for 4 d and then the solvent was removed in vacuo. The residue was dissolved in DMSO (5 mL) and purified by HPLC using a reversed phase C18 column and eluting with a gradient of H₂O:CH₃CN:CF₃CO₂H—90:10:0.1 to 5:95:0.1. The pure, product-containing fractions were combined and concentrated to give the title compound as the trifluoroacetate salt. MS: m/z—572 (M+1).

### Step. D. (7S)-3-{[(2R)-2-(3,5-Difluorophenyl)-2,5,5-trimethyl-6-oxopiperazin-1-yl]methyl}-6,8-dihydrospiro[cyclopenta[g]quinoline-7,3′-pyrrolo[2,3-b]pyridin]-2′(1′H)-one, isomer B

A solution of N-[2-(3,5-difluorophenyl)-2-({[(7S)-2′-oxo-1′,2′,6,8-tetrahydrospiro[cyclopenta[g]quinoline-7,3′-pyrrolo[2,3-b]pyridin]-3-yl]methyl}amino)propyl]-2-methylalanine from Step D (150 mg, 0.262 mmol), EDC (60.4 mg, 0.315 mmol), HOST (48.2 mg, 0.315 mmol), and DIEA (0.229 mL, 1.31 mmol) in DMF (5 mL) was stirred for 16 h. The reaction mixture was diluted with saturated aqueous NaHCO₃ (20 mL) and extracted with CH₂Cl₂ (3×10 mL). The combined organic extracts were dried over Na₂SO₄, filtered, and concentrated in vacuo. The crude product was purified by silica gel chromatography, eluting with a gradient of CH₂Cl₂:MeOH:NH₄OH—100:0:0 to 90:10:1, to give the title compound as a mixture of diastereomers. The mixture of diastereomers were resolved by HPLC, utilizing a Chiralpak AS-H column and eluting with MeOH:CO₂—20:80. The first major peak to elute was (7S)-3-{[(2R)-2-(3,5-Difluorophenyl)-2,5,5-trimethyl-6-oxopiperazin-1-yl]methyl}-6,8-dihydrospiro[cyclopenta[g]quinoline-7,3′-pyrrolo[2,3-b]pyridin]-2′(1′H)-one, isomer A, and the second major peak to elute was (7S)-3-{[(2R)-2-(3,5-Difluorophenyl)-2,5,5-trimethyl-6-oxopiperazin-1-yl]methyl}-6,8-dihydrospiro[cyclopenta[g]quinoline-7,3′-pyrrolo[2,3-b]pyridin]-2′(1′H)-one, isomer B, the title compound. MS: m/z=554 (M+1). HRMS: m/z=554.2365; calculated m/z=554.2362 for C₃₂H₃₀F₂N₅O₂.

Essentially following analogous procedures to those outlined for Intermediates 1-39 the compounds in Table 3 were prepared. The requisite starting materials were described herein, commercially available, described in the literature, or readily synthesized by one skilled in the art of organic synthesis.

TABLE 3

| Intermediate | Structure | MS (M + 1) |
|---|---|---|
| 40 | 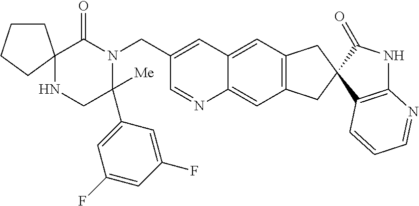 | 580 |
| 41 | 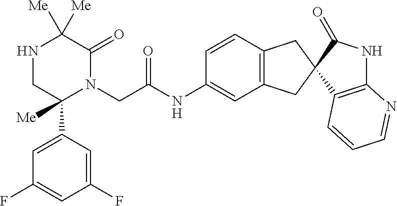 | 546 |
| 42 | 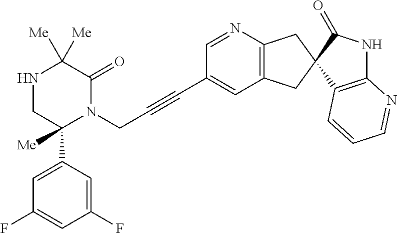 | 528 |
| 43 | 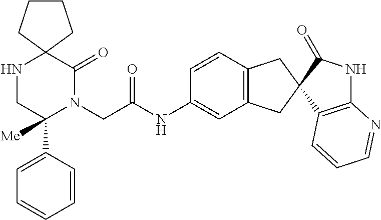 | 536 |
| 44 | 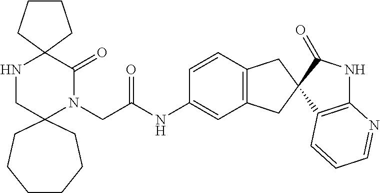 | 528 |
| 45 | 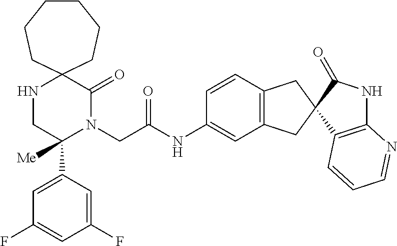 | 600 |
| 46 | 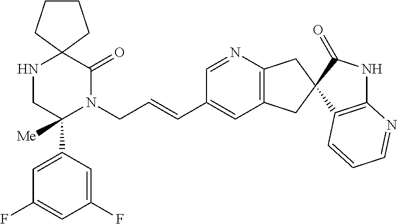 | 556 |
| 47 | 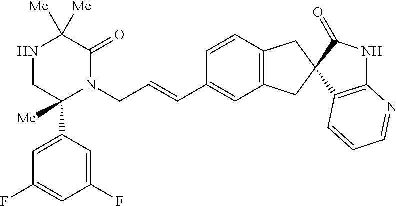 | 529 |
| 48 | 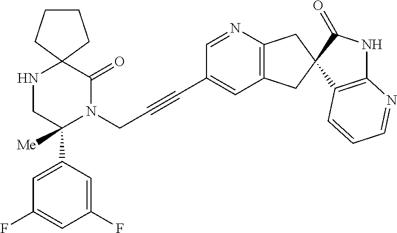 | 554 |
| 49 | 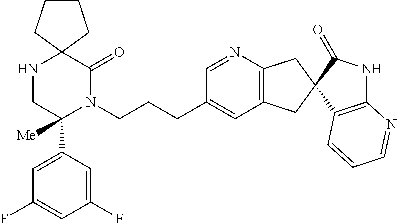 | 558 |
| 50 | 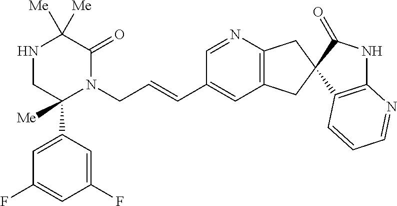 | 530 |

### Example 1

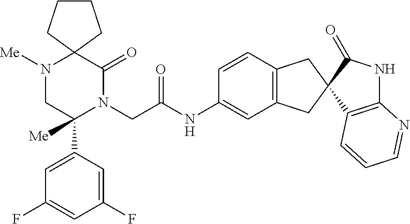

### 2-[(8R)-8-(3,5-Difluorophenyl)-6,8-dimethyl-10-oxo-6,9-diazaspiro[4.5]dec-9-yl]-N-[(2R)-2′-oxo-1,1′,2′,3-tetrahydrospiro[indene-2,3′-pyrrolo[2,3-b]pyridin]-5-yl]acetamide

To a stirred solution of 2-[(8R)-8-(3,5-difluorophenyl)-8-methyl-10-oxo-6,9-diazaspiro[4.5]dec-9-yl]-N-[(2R)-2′-oxo-1,1′,2′,3-tetrahydrospiro[indene-2,3′-pyrrolo[2,3-b]pyridin]-5-yl]acetamide (30 mg, 0.047 mmol, described in Intermediate 38) and AcOH (0.008 mL, 0.14 mmol) in MeOH (1 mL) was added formaldehyde (0.0076 mL, 37 wt % in H₂O, 0.093 mmol). The mixture was stirred for 5 min and NaCNBH₃ (3.5 mg, 0.056 mmol) was added. The reaction mixture was stirred at ambient temperature for 2 h, then poured into aqueous NaHCO₃ (4 mL) and extracted with EtOAc (10 mL). The organic layer was dried over Na₂SO₄, filtered, and concentrated in vacuo. The crude product was dissolved in MeOH (2 mL) and K₂CO₃ (5 mg) was added. The mixture was stirred at ambient temperature for 2 h, then poured into aqueous NaHCO₃ (5 mL) and extracted with EtOAc (10 mL). The organic layer was dried over Na₂SO₄, filtered, and concentrated in vacuo. The crude product was purified by silica gel chromatography, eluting with a gradient of CH₂Cl₂:MeOH—100:0 to 90:10, to give a crude sample of the title compound. Further purification was achieved by HPLC using a reversed phase C18 column and eluting with a gradient of H₂O:CH₃CN:CF₃CO₂H—90:10:0.1 to 5:95:0.1. The desired fractions were poured into aqueous NaHCO₃ (20 mL) and extracted with EtOAc (40 mL). The organic layer was dried over Na₂SO₄, filtered, and concentrated in vacuo to give the title compound. MS: m/z=586 (M+1). HRMS: m/z=586.2646; calculated m/z=586.2624 for C₃₃H₃₄F₂N₅O₃.

Essentially following analogous procedures to those outlined for Example 1, as well as other standard methodology, the compounds in Table 4 were prepared. The requisite starting materials were described herein, commercially available, described in the literature, or readily synthesized by one skilled in the art of organic synthesis.

TABLE 4

| Example | Structure | MS (M + 1) |
|---|---|---|
| 2 | 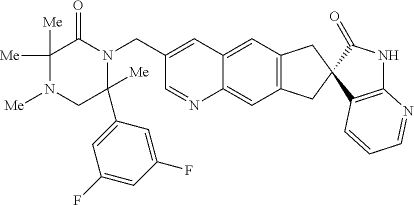 | 568 |
| 3 | 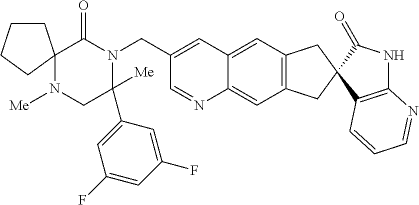 | 594 |
| 4 | 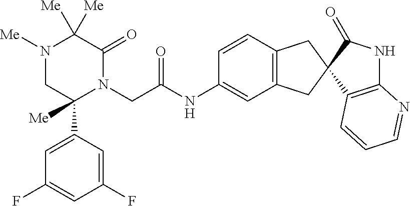 | 560 |
| 5 | 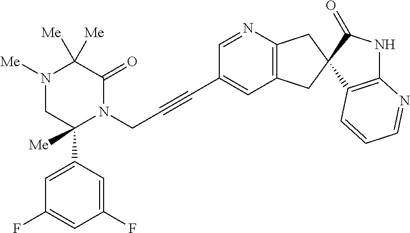 | 542 |
| 6 | 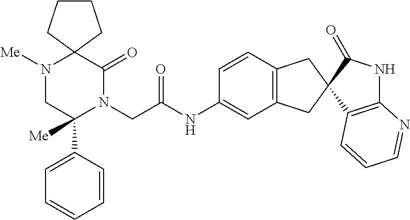 | 550 |
| 7 | 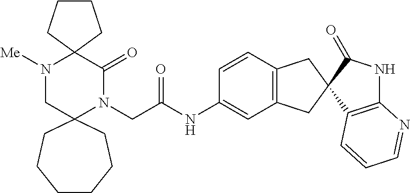 | 542 |
| 8 | 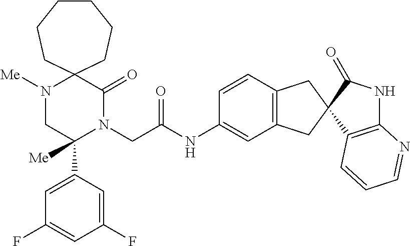 | 614 |
| 9 | 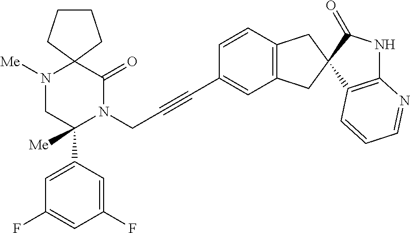 | 567 |
| 10 | 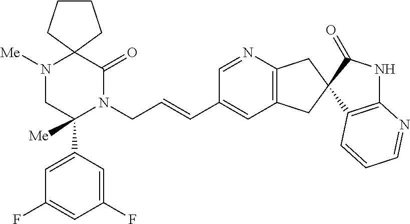 | 570 |
| 11 | 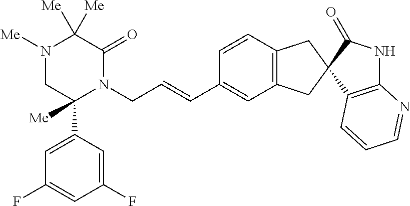 | 543 |
| 12 | 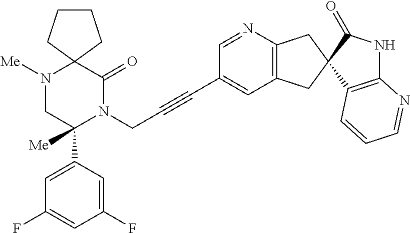 | 568 |
| 13 | 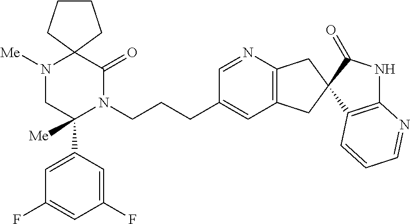 | 572 |
| 14 | 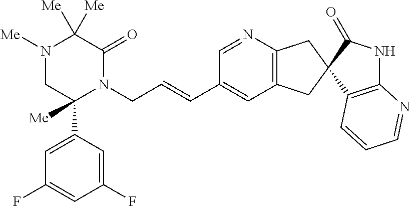 | 544 |

### [¹¹C]-Example 1

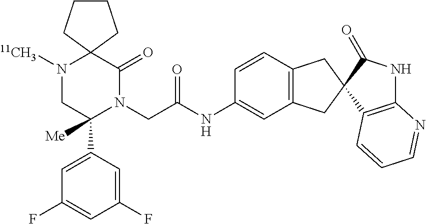

### [¹¹C]-2-[(8R)-8-(3,5-Difluorophenyl)-6,8-dimethyl-10-oxo-6,9-diazaspiro[4.5]dec-9-yl]-N-[(2R)-2′-oxo-1,1′,2′,3-tetrahydrospiro[indene-2,3′-pyrrolo[2,3-b]pyridin]-5-yl]acetamide

[¹¹C]CO₂ was produced by Siemens Biomarker Solutions, Inc. (North Wales, Pa.) using a Siemens RDS-111 cyclotron. An N-14 gas target containing 5% oxygen was irradiated with an 11 MeV proton beam generating [¹¹C]CO₂. The [¹¹C]CO₂ was converted to [¹¹C]methyl triflate using a GE Medical Systems TRACERIab FXc system. Radiochemical procedures were carried out using a Gilson (Worthington, Ohio) 233XL liquid handler.

[¹¹C]Methyl triflate (˜300 mCi) was trapped in a solution of 2-[(8R)-8-(3,5-difluorophenyl)-8-methyl-10-oxo-6,9-diazaspiro[4.5]dec-9-yl]N-[(2R)-2′-oxo-1,1′,2′,3-tetrahydrospiro[indene-2,3′-pyrrolo[2,3-b]pyridin]-5-yl]acetamide (0.2-0.5 mg, described in Intermediate 38) in acetone (0.25 mL) at room temperature for 1 min. This mixture was then diluted with 10 mM Na₂HPO₄ solution (0.5 mL) and purified by preparative HPLC (Phenomenex Gemini C18 column, 10×150 mm, 5 μm). The solvent system used was 60:40 EtOH:5 mM Sodium Citrate 3 ml/min with a retention time of ˜8 min. The peak corresponding to the PET tracer of [¹¹C]-Example 1 was collected and diluted with saline to give 87 mCi of the PET tracer of [¹¹C]-Example 1 with a specific activity of 1832 Ci/mmol and a radiochemical purity of >97% (n=10). The specific activity and radiochemical purity of the PET tracer of [¹¹C]-Example 1 was determined by counting an aliquot in a dose calibrator and determining the mass by analytical HPLC (C18 XTerra RP18, 4.6×150 mm, 5 μm) against an authentic standard. The solvent system used was 30:70 acetonitrile (solvent A): 0.1% aq. TFA (solvent B) at 1 ml/min, 254 nm, with retention time of 5.5 min.

When determining appropriate ligands for candidate PET tracers, several criteria should be considered. In order to achieve a useful specific signal, the ligand must have low nonspecific binding, which is often related to the ligand's lipophilicity. Log P (octanol/water partition coefficient) at physiological pH ˜7.4 is often used as a surrogate measure of the lipophilicity of a ligand, and Log P values <3.5 are preferred for PET tracers. For a CNS target such as CGRP, the ligand must penetrate the blood brain barrier (BBB), which is also dependent upon its lipophilicity. A log P of >1 is desired, such that the ligand is not too polar to passively defuse cross the BBB. Additionally, the BBB possesses efflux pumps which can prevent compounds from effectively accumulating in the brain, of which P-glycoprotein (P-gp) is a key efflux pump. Therefore, a PET tracer should not be a good substrate for P-gp. Furthermore, of primary consideration is the ratio of the ligand's affinity (K_{d} or other relevant measure such as Kᵢ) to the concentration (Bₘₐₓ) of the target, in this case CGRP. Preliminary in vitro saturation binding studies in rhesus monkey cerebellum tissue homogenate indicated the Bₘₐₓ of CGRP binding sites were ˜20 nM (unpublished data). We generally consider PET tracers with a Bₘₐₓ/K_{d} ratio of >10 to have a high probability of providing a specific signal in vivo. Therefore, a ligand with a K_{d}<2 nM should be adequate to provide a signal in vivo. Finally, the ligand must have structural feature(s) suitable for facile incorporation of a positron emitting isotope, such as ¹⁸F or ¹¹C, with high specific activity.

The compound of Example 1 possesses a high affinity and selectivity for the CGRP receptor and suitable physical properties. The octanol/water partition coefficient (Log P) of the compound of Example 1 was determined by partitioning the compound between octanol and phosphate buffer at pH 7.4 and measuring the concentration of the compound in each layer by LC-MS/MS. The compound of Example 1 was determined to possess a suitable log P=3.38. The compound of Example 1 was tested in the receptor binding and functional cell-based assays carried out in the presence and absence of human serum. The compound of Example 1 has a Kᵢ on the human CGRP receptor of 0.039 nM vs. [¹²⁵I]CGRP and has an IC₅₀ in the cAMP functional assay in the presence of 50% human serum of 0.21 nM. The compound of Example 1 is selective for the CGRP receptor, CLR-RAMP1, relative to the adrenomedullin 2 receptor, CLR-RAMP3 (Kᵢ=211 nM).

The compound of Example 1 was evaluated against the related amylin 1 (AMY₁; CTR/RAMP1) and amylin 3 (AMY₃; CTR/RAMP3) receptors. The compound of Example 1 displays significant activity on the human AMY₁ receptor (Kᵢ=0.6 nM) and is selective versus the human AMY₃ receptor (Kᵢ=212 nM). Although the compound of Example 1 binds with high affinity to the related AMY₁ receptor, this is not anticipated to negatively impact the utility of this molecule as a suitable PET tracer. The cerebellum, which displays a high density of CGRP receptor binding sites, does not contain appreciable levels of amylin binding sites. Since the methods used to quantify CGRP receptor occupancy rely primarily on the cerebellar region, the affinity of the compound of Example 1 for AMY₁ receptor should not interfere with the use of the PET tracer to accurately determine CGRP receptor occupancy.

Additionally, the compound of Example 1 is brain penetrant, possesses good membrane permeability and lacks susceptibility for transport by the P-gp drug effex pump. The bidirectional transport of the compound of Example 1 was evaluated across mono-layers of LLC-PK1 cells over-expressing human P-glycoprotein (Ohe et al., 2003). The compound of Example 1 was not a substrate for human P-glycoprotein (P-gp) and was a borderline substrate for rat P-gp with B−A/A−B ratios of 1.7 and 3.1 for human and rat (at 5 μM), respectively. Testing at 1, 0.5, and 0.1 μM for both human and African green monkey P-gp activity resulted in B−A/A−B ratios that indicated the compound was a weak substrate for P-gp in both species (3, 2.9, and 3.5 for human, 3.9, 4.1, and 5.4 for African green monkey, respectively). The compound of Example 1 displayed high passive permeability with an average apparent permeability coefficient (Pₐₚₚ) of 26×10⁻⁶ cm/s.

Baseline PET scans in rhesus monkey have confirmed the uptake and expected regional distribution of the PET tracer of [¹¹C]-Example 1 in the brain. The highest uptake was in the cerebellum and brain stem, consistent with the in vitro autoradiography results. In vivo occupancy studies were carried out with the compound of Example 1 to examine the utility of the PET tracer of [¹¹C]-Example 1 to establish a drug plasma level/CGRP receptor occupancy relationship. To establish steady plasma levels of drug, the compound of Example 1 was administered as an IV bolus plus constant infusion starting 60 minutes before the PET tracer of [¹¹C]-Example 1 injection; drug infusion was continued for the duration of the scan. Dynamic PET studies were acquired for 120-min following the PET tracer of [¹¹C]-Example 1 IV bolus administration. CGRP receptor availability was estimated using data-driven methods to describe the PET tracer of [¹¹C]-Example 1 plasma-brain kinetics. For each study, the PET tracer of [¹¹C]-Example 1 plasma concentration was obtained from the measurement of total radioactivity in arterial plasma with correction for the fraction of intact tracer as determined by HPLC analysis.

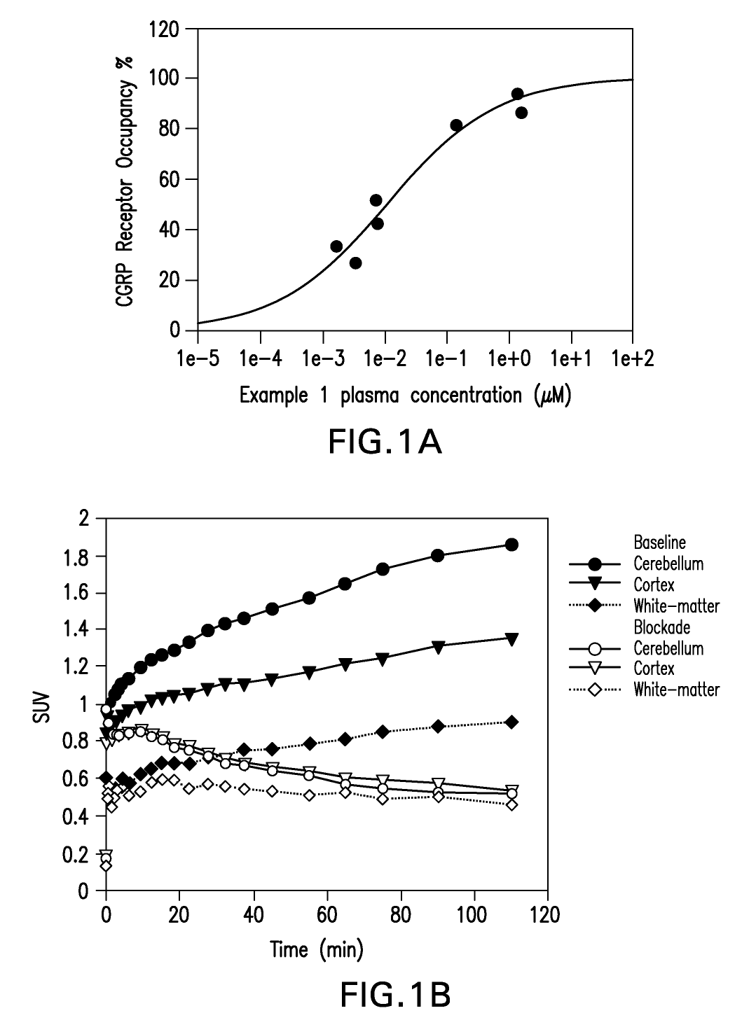

Self-blockade with high levels of the compound of Example 1 resulted in substantial reduction of the PET tracer of [¹¹C]-Example 1 uptake in all brain regions with a specific signal of ˜2:1 in the cerebellum. The absence of a reference region (devoid of CGRP receptor) requires the application of tracer kinetic modeling techniques using the PET tracer of [¹¹C]-Example 1 arterial plasma concentration in order to accurately quantify CGRP receptor occupancy in rhesus monkey. Application of these modeling techniques was useful in determining a dose/occupancy relationship for unlabeled compound Example 1 (FIG. 1). Data were described using the Hill equation, CGRP occupancy 100% yⁿ/[Occ₅₀ⁿ+yⁿ], where y corresponds to the average compound of Example 1 plasma level during the PET scan. The estimated parameters (±SE) were: n=0.49±0.07 and Occ₅₀=11±3 nM.

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention.

1. A compound of Formula I 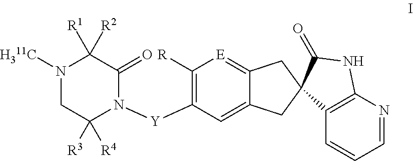
or a pharmaceutically acceptable salt thereof, wherein:
E is N or CH;
R is H and Y is a linker selected from the group consisting of: 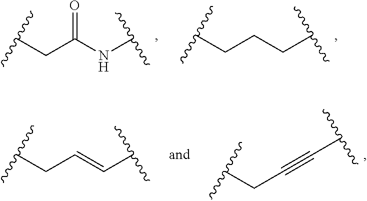
or R and Y together represent 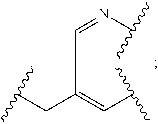
R¹ and R² are independently C₁₋₄alkyl, or R¹ and R² are joined together with the atom to which they are attached to form cyclopentyl, cyclohexyl or cycloheptyl;
and
R³ is hydrogen or methyl and R⁴ is phenyl optionally substituted with 1 to 5 halo groups,
or R³ and R⁴ are joined together with the atom to which they are attached to form cyclopentyl, cyclohexyl or cycloheptyl.

2. The compound of claim 1 according to Formula Ia 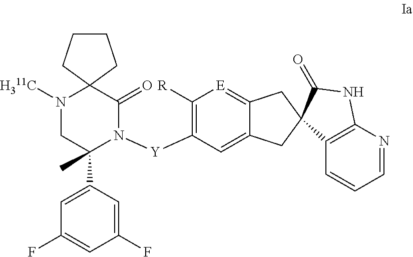
or a pharmaceutically acceptable salt thereof.

3. The compound of claim 1 selected from: 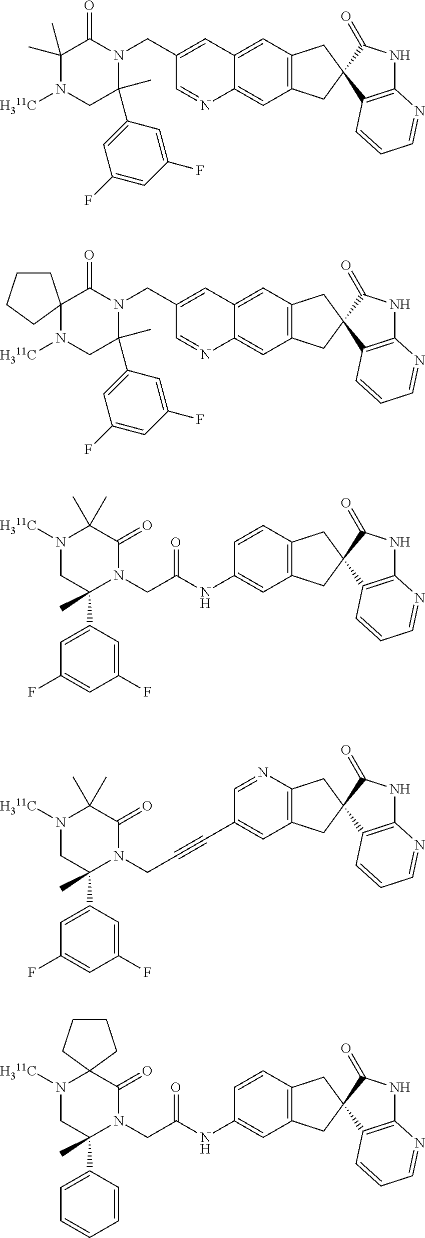 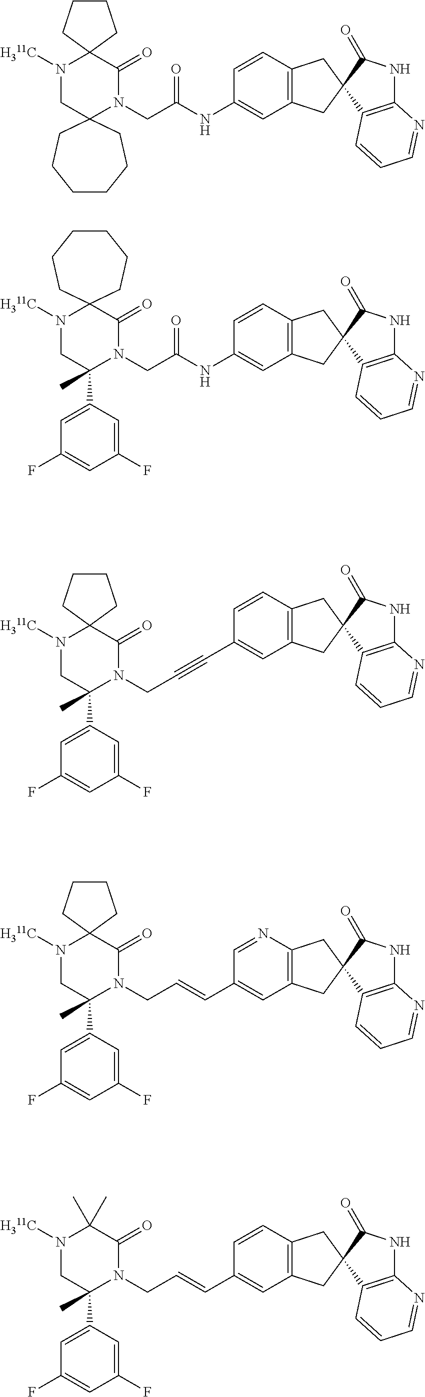 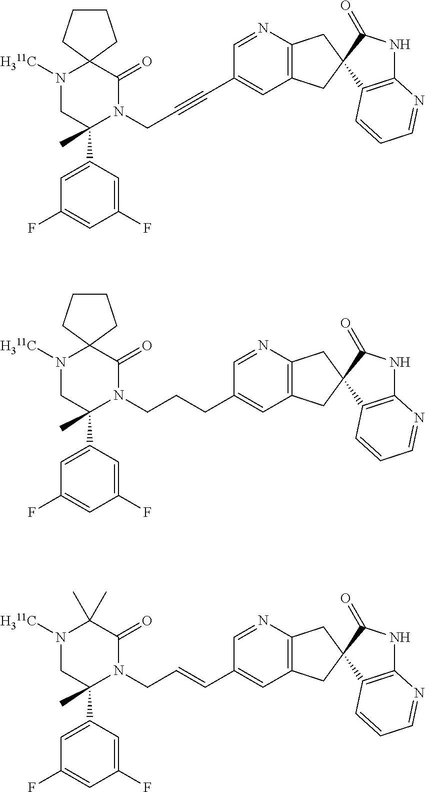
or a pharmaceutically acceptable salt of any of the foregoing.

4. The compound of claim 1 which is 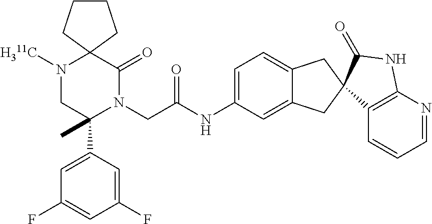
or a pharmaceutically acceptable salt thereof.

5. A radiopharmaceutical composition which comprises the compound of claim 1 and a pharmaceutically acceptable carrier or excipient.

6. (canceled)

7. A method for the quantitative imaging of CGRP receptors in a mammal which comprises administering to the mammal an effective amount of the compound of claim 1, and obtaining an image of CGRP receptors in the mammal using positron emission tomography.

8. The method of claim 7 wherein the CGRP receptors are in the brain of a mammal.

9. The method of claim 7 wherein the CGRP receptors are in tissues bearing CGRP receptors in a mammal.

10. (canceled)

11. A radiopharmaceutical composition which comprises the compound of claim 4 and a pharmaceutically acceptable carrier or excipient.

12.-15. (canceled)

16. A compound of Formula I 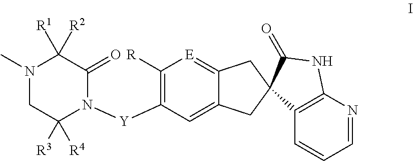
or a pharmaceutically acceptable salt thereof, wherein:
E is N or CH;
R is H and Y is a linker selected from the group consisting of: 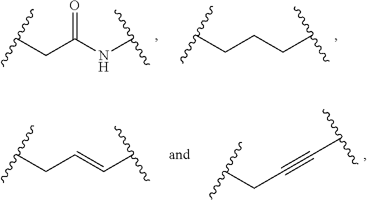
or R and Y together represent 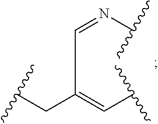
R¹ and R² are independently C₁₋₄alkyl, or R¹ and R² are joined together with the atom to which they are attached to form cyclopentyl, cyclohexyl or cycloheptyl;
and
R³ is hydrogen or methyl and R⁴ is phenyl optionally substituted with 1 to 5 halo groups,
or R³ and R⁴ are joined together with the atom to which they are attached to form cyclopentyl, cyclohexyl or cycloheptyl.

17. The compound of claim 16 according to Formula Ia 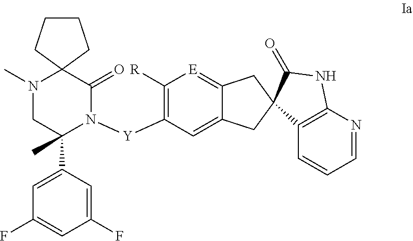
or a pharmaceutically acceptable salt thereof.

18. The compound of claim 16 selected from: 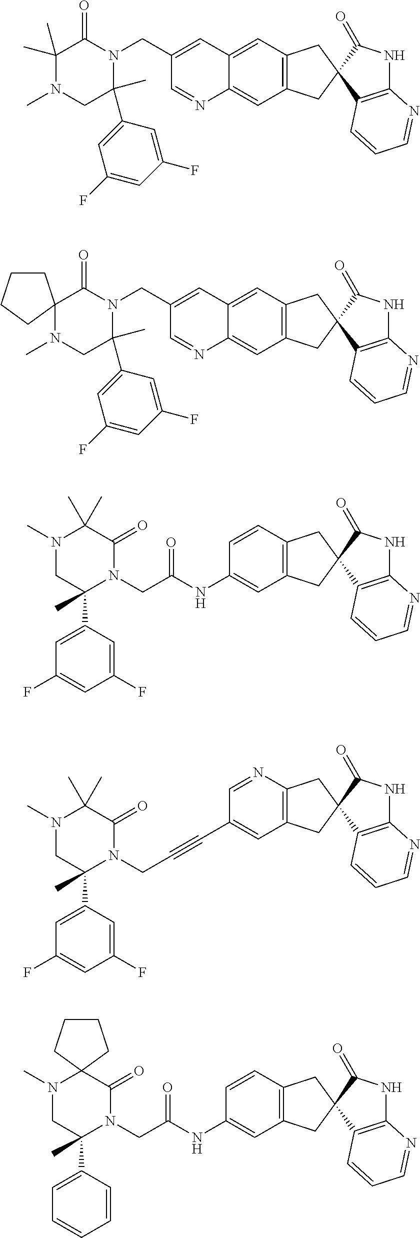 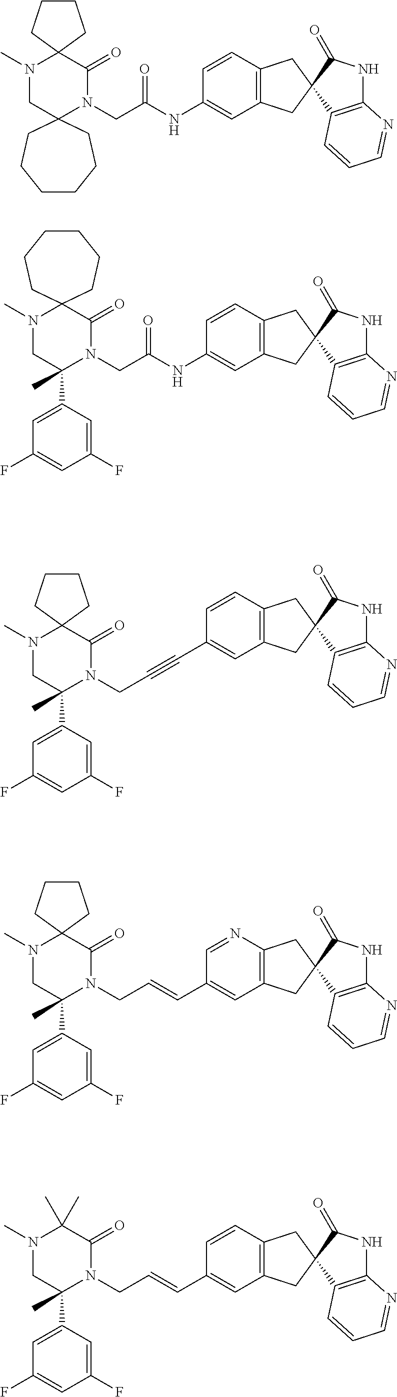 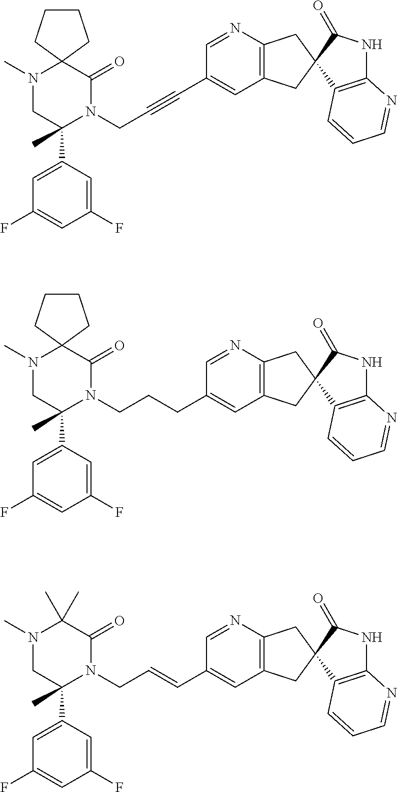
or a pharmaceutically acceptable salt of any of the foregoing.

19. The compound of claim 16 which is 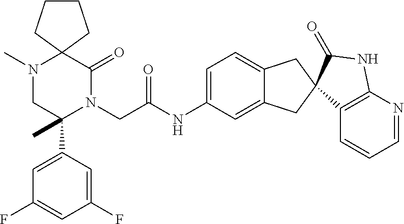
or a pharmaceutically acceptable salt thereof.
